(19) United States
(12) Patent Application Publication
(10) Pub. No.: US 2022/0073638 A1
(43) Pub. Date: Mar. 10, 2022

(54) METHODS AND PHARMACEUTICAL COMPOSITION FOR THE TREATMENT OF CANCERS RESISTANT TO IMMUNE CHECKPOINT THERAPY

(71) Applicants: INSERM (INSTITUT NATIONAL DE LA SANTÉ ET DE LA RECHERCHE MÉDICALE, Paris (FR); UNIVERSITÉ D'AIX MARSEILLE, Marseille Cedex 07 (FR); CENTRE NATIONAL DE LA RECHERCHE SCIENTIFIQUE (CNRS), Paris (FR); Aarhus Universitet, Aarhus C (DK)

(72) Inventors: Toby Lawrence, London (GB); Søren Kragh Moestrup, Aarhus N (DK); Anders Etzerodt, Hinnerup (DK)

(21) Appl. No.: 17/276,432

(22) PCT Filed: Sep. 18, 2019
(86) PCT No.: PCT/EP2019/075080

§ 371 Date: Mar. 15, 2021

(30) Foreign Application Priority Data
Sep. 19, 2018 (EP) 18306219.9

(51) Int. Cl. C07K 16/28

(52) U.S. Cl. CPC C07K 16/2896; A61K 2039/505

(57) ABSTRACT

Recent advances in the understanding of macrophage biology has revealed that tumor-associated macrophages are very heterogeneous and that several distinct subsets coexist in the tumor microenvironment. These subsets differ not only in terms of expression profile and origin but also in their pro- or anti-tumoral function. Here, the inventors describe a macrophage subset in mouse models of metastatic melanoma that express CD 163. Specific depletion of the CD 163 expressing cells in an anti-PD-1 checkpoint inhibitor resistant melanoma model using cytotoxic lipid nanoparticles conjugated to αCD163 mAh results in a massive infiltration of CD4+ and activated CD8⁺ T-cells. Moreover the inventors show that tumors quickly relapsed with combined treatment with anti-PDI antibodies. Thus the present invention relates to a method of treating a cancer in a subject in need thereof comprising administering to the subject a therapeutically effective combination comprising at least one immune checkpoint inhibitor and an agent capable of depleting the population of CD 163+ tumor associated macrophages.

### FIELD OF THE INVENTION

The present invention relates to methods and pharmaceutical composition for the treatment of cancers resistant to immune checkpoint therapy.

### BACKGROUND OF THE INVENTION

Tumor-associated macrophages (TAM) are the most abundant immune cells found in solid tumors and their important contributions to tumor progression are well documented¹.

Besides their trophic functions, supporting angiogenesis, invasion and metastasis, TAM have also been suggested to inhibit T cell proliferation and activation via release of the immune-suppressive cytokine IL-10² and the local depletion of arginine³ and tryptophan⁴, on which T cells are highly dependent. The important functions of macrophages in relation to tumor progression has already attracted substantial interest in developing new therapeutic strategies for targeting TAM. Strategies currently in clinical trials include, blocking of the chemokine CCL2, or its receptor CCR2, which inhibits TAM recruitment by neutralizing the mobilization of bone marrow-derived monocytes, and targeting the macrophage growth factor receptor CSF1R (M-CSFR; c-FMS; CD115)⁵. While CCL2/CCR2 blockade only targets the recruitment of monocyte-derived macrophages, CSF1 plays an essential role in both the survival and differentiation of tissue-resident macrophages as well as maturation of monocyte-derived macrophage⁶. Indeed, although clinical data is still limited, reduced numbers of TAM has been reported after treatment with monoclonal antibody (mAb) therapy against CSF1R, with potentially interesting therapeutic effects in tenosynovial giant cell tumors^{7,8}.

Despite a strong association between TAM accumulation and poor clinical outcome in the vast majority of clinical studies, certain reports associates TAM numbers or accumulation of specific TAM subsets with a good prognosis. One example is the frequency of HLA-DR⁺ TAM that has been associated with beneficial outcomes in several studies^{9,10}, potentially reflecting their roles in orchestrating protective immune responses¹¹. To this extend, recent studies using paired single cell analysis by mass cytometry and RNA sequencing, have revealed an unprecedented level of diversity within the tumor-infiltrating myeloid cell (TIM) compartment in lung adenocarcinoma and renal cell carcinoma (RCC) patients^{12,13}. In the case of RCC, 17 distinct TAM phenotypes were documented¹². Although we still lack a deeper understanding about the functions of different TAM subsets and their respective contributions to tumor progression, it is tempting to speculate that a selective targeting of TAM subsets that abrogates tumor-promoting mechanisms while preserving innate immune functions may promote anti-tumor immunity and could offer significant clinical benefits.

Expression of CD163 by TAM has been shown to be a particularly strong indicator of poor prognosis in several different cancers. CD163 is a macrophage and monocyte specific transmembrane protein that functions as a scavenger receptor for haptoglobin-hemoglobin complexes, formed upon intravascular haemolysis¹⁴. Expression of CD163 is induced by tumor-promoting cytokines such as IL-6 and IL-10, whereas inflammatory stimuli, including lipopolysaccharide (LPS), TNFα and IFNγ, lead to a rapid downregulation of expression and removal of membrane bound CD163 via proteolytic shedding^{15,16}. This, together with the generation of anti-inflammatory heme metabolites from hemoglobin scavenging, has led to the association of CD163⁺ macrophages with anti-inflammatory functions¹⁵. However, the links between CD163⁺ TAM accumulation and tumor progression are based exclusively on correlations with clinical progression and experimental evidence for specific tumor-promoting functions is still lacking.

The recent development of immune checkpoint inhibitors (ICI), such as anti-PD-1, has had an enormous impact on cancer therapy, particularly in malignant melanoma^{17,18}. The increased expression of PD-1 ligand (PD-L1) on cancer cells suppresses the activation of PD-1-expressing CD8⁺ cytotoxic T cells (CTL)¹⁹. Blocking PD-1/PD-L1 signaling, using an anti-PD-1 or PD-L1 mAbs, thus leads to increased activation of CTL, ultimately resulting in unprecedented rates of tumor regression²⁰. Unfortunately, only a minority of patients respond to ICI therapy and the reasons for this is currently an area of intense research. In addition, a major limitation of ICI therapy is the indiscriminate activation of T cells, which can lead to severe immune-related adverse events making continued treatment impossible^{21,22}. Thus, new therapeutic strategies to enhance anti-tumor immunity that can overcome ICI resistance or ameliorate the severe adverse side effects, are desperately needed.

### SUMMARY OF THE INVENTION

The present invention relates to methods and pharmaceutical composition for the treatment of cancers resistant to immune checkpoint therapy. In particular, the present invention is defined by the claims.

### DETAILED DESCRIPTION OF THE INVENTION

Recent advances in the understanding of macrophage biology has revealed that tumor-associated macrophages are very heterogeneous and that several distinct subsets coexist in the tumor microenvironment. These subsets differ not only in terms of expression profile and origin but also in their pro- or anti-tumoral function. Here, the inventors describe a macrophage subset in mouse models of metastatic melanoma that express CD163 and immune-modulatory cytokines such as IL10, Ido1 and Lgals1. Specific depletion of the CD163 expressing cells in an anti-PD-1 checkpoint inhibitor resistant melanoma model using cytotoxic lipid nanoparticles conjugated to αCD163 mAb results in a massive infiltration of CD4⁺ and activated CD8⁺ T-cells. The specific depletion of CD163⁺ TAM alone allows an accumulation of CD163^{neg} inflammatory macrophages that in combination with activated T-cells drives an anti-tumor immune response and tumor regression. Taken together, the data reveal that CD163⁺ macrophages have a strong immune-suppressive function and that loss of CD163⁺ macrophages results in a re-education of the tumor immune-microenvironment. This suggest that CD163⁺ macrophages are pivotal for maintaining a pro-tumoral tumor-immune microenvironment and that targeting of this populations could offer an attractive therapeutic target in immune checkpoint inhibitor resistant tumors.

As used herein, the term “CD163” (Cluster of Differentiation 163) also known as M130 MM130 or “SCARI1 has its general meaning in the art and refers to a protein that in humans is encoded by the CD163 gene [Gene ID: 9332]. CD163 is exclusively expressed in monocytes and macrophages. It functions as an acute phase-regulated receptor involved in the clearance and endocytosis of hemoglobin/haptoglobin complexes by macrophages, and may thereby protect tissues from free hemoglobin-mediated oxidative damage. This protein may also function as an innate immune sensor for bacteria and inducer of local inflammation. The molecular size is 130 kDa. The receptor belongs to the scavenger receptor cysteine rich family type B and consists of a 1048 amino acid residues extracellular domain, a single transmembrane segment and a cytoplasmic tail with several splice variants. An exemplary human amino acid sequence is represented by SEQ ID NO:1. The extracellular domain of CD163 ranges from the amino acid residue at position 42 to the amino acid residue 1050 at position in SEQ ID NO:1.

>sp|Q86VB7|C163A_HUMAN Scavenger receptor
cysteine-rich type 1 protein M130 OS = Homo
sapiens OX = 9606 GN = CD163 PE = 1 SV = 2.
The extracellular domains is shown as
underlined.
SEQ ID NO: 1
MSKLRMVLLEDSGSADFRRHFVNLSPFTITVVLLLSACFVTSSLGGTDKE
LRLVDGENKCSGRVEVKVQEEWGTVCNNGWSMEAVSVICNQLGCPTAIKA
PGWANSSAGSGRIWMDHVSCRGNESALWDCKHDGWGKHSNCTHQQDAGVT
CSDGSNLEMRLTRGGNMCSGRIEIKFQGRWGTVCDDNFNIDHASVICRQL
ECGSAVSFSGSSNFGEGSGPIWFDDLICNGNESALWNCKHQGWGKHNCDH
AEDAGVICSKGADLSLRLVDGVTECSGRLEVRFQGEWGTICDDGWDSYDA
AVACKQLGCPTAVTAIGRVNASKGFGHIWLDSVSCQGHEPAIWQCKHHEW
GKHYCNHNEDAGVTCSDGSDLELRLRGGGSRCAGTVEVEIQRLLGKVCDR
GWGLKEADVVCRQLGCGSALKTSYQVYSKIQATNTWLFLSSCNGNETSLW
DCKNWQWGGLTCDHYEEAKITCSAHREPRLVGGDIPCSGRVEVKHGDTWG
SICDSDFSLEAASVLCRELQCGTVVSILGGAHFGEGNGQIWAEEFQCEGH
ESHLSLCPVAPRPEGTCSHSRDVGVVCSRYTEIRLVNGKTPCEGRVELKT
LGAWGSLCNSHWDIEDAHVLCQQLKCGVALSTPGGARFGKGNGQIWRHMF
HCTGTEQHMGDCPVTALGASLCPSEQVASVICSGNQSQTLSSCNSSSLGP
TRPTIPEESAVACIESGQLRLVNGGGRCAGRVEIYHEGSWGTICDDSWDL
SDAHVVCRQLGCGEAINATGSAHFGEGTGPIWLDEMKCNGKESRIWQCHS
HGWGQQNCRHKEDAGVICSEFMSLRLTSEASREACAGRLEVFYNGAWGTV
GKSSMSETTVGVVCRQLGCADKGKINPASLDKAMSIPMWVDNVQCPKGPD
TLWQCPSSPWEKRLASPSEETWITCDNKIRLQEGPTSCSGRVEIWHGGSW
GTVCDDSWDLDDAQVVCQQLGCGPALKAFKEAEFGQGTGPIWLNEVKCKG
NESSLWDCPARRWGHSECGHKEDAAVNCTDISVQKTPQKATTGRSSRQSS
FIAVGILGVVLLAIFVALFFLTKKRRQRQRLAVSSRGENLVHQIQYREMN
SCLNADDLDLMNSSENSHESADFSAAELISVSKFLPISGMEKEAILSHTE
KENGNL

As used herein, the term “tumor associated macrophage” or “TAM” has its general meaning in the art and is intended to describe a type of cell belonging to the macrophage lineage. They are found in close proximity or within tumor masses. TAMs are derived from circulating monocytes or resident tissue macrophages, which form the major leukocytic infiltrate found within the stroma of many tumor types. Accordingly, the term “CD163+ tumor associated macrophages” refers to a subset of TAM characterized by the expression of CD163. In some embodiments, the population of CD163+ tumor associated macrophages of the present invention is further characterized by the expression of and immune-modulatory cytokines such as IL10, Ido1 and Lgals1.

Accordingly, the first object of the present invention relates to a method of increasing the amount of tumor infiltrating CD8+ T cells in a patient suffering from cancer comprising administering to the patient a therapeutically effective amount of an agent capable of depleting the population of CD163+ tumor associated macrophages.

As used herein, the term “CD8+ T cell” has its general meaning in the art and refers to a subset of T cells which express CD8 on their surface. They are MHC class I-restricted, and function as cytotoxic T cells. “CD8+ T cells” are also called cytotoxic T lymphocytes (CTL), T-killer cells, cytolytic T cells, or killer T cells. CD8 antigens are members of the immunoglobulin supergene family and are associative recognition elements in major histocompatibility complex class I-restricted interactions. As used herein, the term “tumor infiltrating CD8+ T cell” refers to the pool of CD8+ T cells of the patient that have left the blood stream and have migrated into a tumor.

As used herein, the term “cancer” has its general meaning in the art and includes, but is not limited to, solid tumors and blood-borne tumors. The term cancer includes diseases of the skin, tissues, organs, bone, cartilage, blood and vessels. The term “cancer” further encompasses both primary and metastatic cancers. Examples of cancers that may be treated by methods and compositions of the invention include, but are not limited to, cancer cells from the bladder, blood, bone, bone marrow, brain, breast, colon, esophagus, gastrointestinal tract, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; Leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, the subject suffers from melanoma. As used herein, “melanoma” refers to a condition characterized by the growth of a tumor arising from the melanocytic system of the skin and other organs. Most melanocytes occur in the skin, but are also found in the meninges, digestive tract, lymph nodes and eyes. When melanoma occurs in the skin, it is referred to as cutaneous melanoma. Melanoma can also occur in the eyes and is called ocular or intraocular melanoma. Melanoma occurs rarely in the meninges, the digestive tract, lymph nodes or other areas where melanocytes are found. 40-60% of melanomas carry an activating mutation in the gene encoding the serine-threonine protein kinase B-RAF (BRAF). Among the BRAF mutations observed in melanoma, over 90% are at codon 600, and among these, over 90% are a single nucleotide mutation resulting in substitution of glutamic acid for valine (BRAFV600E).

A further object of the present invention relates to a method of treating a cancer in a subject in need thereof comprising administering to the subject a therapeutically effective combination comprising at least one immune checkpoint inhibitor and an agent capable of depleting the population of CD163+ tumor associated macrophages.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the term “immune checkpoint inhibitor” has its general meaning in the art and refers to any compound inhibiting the function of an immune inhibitory checkpoint protein. As used herein the term “immune checkpoint protein” has its general meaning in the art and refers to a molecule that is expressed by T cells in that either turn up a signal (stimulatory checkpoint molecules) or turn down a signal (inhibitory checkpoint molecules). Immune checkpoint molecules are recognized in the art to constitute immune checkpoint pathways similar to the CTLA-4 and PD-1 dependent pathways (see e.g. Pardoll, 2012. Nature Rev Cancer 12:252-264; Mellman et al., 2011. Nature 480:480-489). Examples of inhibitory checkpoint molecules include A2AR, B7-H3, B7-H4, BTLA, CTLA-4, CD277, IDO, KIR, PD-1, LAG-3, TIM-3 and VISTA. Inhibition includes reduction of function and full blockade. Preferred immune checkpoint inhibitors are antibodies that specifically recognize immune checkpoint proteins. A number of immune checkpoint inhibitors are known and in analogy of these known immune checkpoint protein inhibitors, alternative immune checkpoint inhibitors may be developed in the (near) future. The immune checkpoint inhibitors include peptides, antibodies, nucleic acid molecules and small molecules. Examples of immune checkpoint inhibitor includes PD-1 antagonist, PD-L1 antagonist, PD-L2 antagonist CTLA-4 antagonist, VISTA antagonist, TIM-3 antagonist, LAG-3 antagonist, IDO antagonist, KIR2D antagonist, A2AR antagonist, B7-H3 antagonist, B7-H4 antagonist, and BTLA antagonist.

In some embodiments, PD-1 (Programmed Death-1) axis antagonists include PD-1 antagonist (for example anti-PD-1 antibody), PD-L1 (Programmed Death Ligand-1) antagonist (for example anti-PD-L1 antibody) and PD-L2 (Programmed Death Ligand-2) antagonist (for example anti-PD-L2 antibody). In some embodiments, the anti-PD-1 antibody is selected from the group consisting of MDX-1106 (also known as Nivolumab, MDX-1106-04, ONO-4538, BMS-936558, and Opdivo®), Merck 3475 (also known as Pembrolizumab, MK-3475, Lambrolizumab, Keytruda®, and SCH-900475), and CT-011 (also known as Pidilizumab, hBAT, and hBAT-1). In some embodiments, the PD-1 binding antagonist is AMP-224 (also known as B7-DCIg). In some embodiments, the anti-PD-L1 antibody is selected from the group consisting of YW243.55.S70, MPDL3280A, MDX-1105, and MEDI4736. MDX-1105, also known as BMS-936559, is an anti-PD-L1 antibody described in WO2007/005874. Antibody YW243.55. S70 is an anti-PD-L1 described in WO 2010/077634 Ai. MEDI4736 is an anti-PD-L1 antibody described in WO2011/066389 and US2013/034559. MDX-1106, also known as MDX-1106-04, ONO-4538 or BMS-936558, is an anti-PD-1 antibody described in U.S. Pat. No. 8,008,449 and WO2006/121168. Merck 3745, also known as MK-3475 or SCH-900475, is an anti-PD-1 antibody described in U.S. Pat. No. 8,345,509 and WO2009/114335. CT-011 (Pidizilumab), also known as hBAT or hBAT-1, is an anti-PD-1 antibody described in WO2009/101611. AMP-224, also known as B7-DCIg, is a PD-L2-Fc fusion soluble receptor described in WO2010/027827 and WO2011/066342. Atezolimumab is an anti-PD-L1 antibody described in U.S. Pat. No. 8,217,149. Avelumab is an anti-PD-L1 antibody described in US 20140341917. CA-170 is a PD-1 antagonist described in WO2015033301 & WO2015033299. Other anti-PD-1 antibodies are disclosed in U.S. Pat. No. 8,609,089, US 2010028330, and/or US 20120114649. In some embodiments, the PD-1 inhibitor is an anti-PD-1 antibody chosen from Nivolumab, Pembrolizumab or Pidilizumab. In some embodiments, PD-L1 antagonist is selected from the group comprising of Avelumab, BMS-936559, CA-170, Durvalumab, MCLA-145, SP142, STI-A1011, STIA1012, STI-A1010, STI-A1014, A110, KY1003 and Atezolimumab and the preferred one is Avelumab, Durvalumab or Atezolimumab.

In some embodiments, CTLA-4 (Cytotoxic T-Lymphocyte Antigen-4) antagonists are selected from the group consisting of anti-CTLA-4 antibodies, human anti-CTLA-4 antibodies, mouse anti-CTLA-4 antibodies, mammalian anti-CTLA-4 antibodies, humanized anti-CTLA-4 antibodies, monoclonal anti-CTLA-4 antibodies, polyclonal anti-CTLA-4 antibodies, chimeric anti-CTLA-4 antibodies, MDX-010 (Ipilimumab), Tremelimumab, anti-CD28 antibodies, anti-CTLA-4 adnectins, anti-CTLA-4 domain antibodies, single chain anti-CTLA-4 fragments, heavy chain anti-CTLA-4 fragments, light chain anti-CTLA-4 fragments, inhibitors of CTLA-4 that agonize the co-stimulatory pathway, the antibodies disclosed in PCT Publication No. WO 2001/014424, the antibodies disclosed in PCT Publication No. WO 2004/035607, the antibodies disclosed in U.S. Publication No. 2005/0201994, and the antibodies disclosed in granted European Patent No. EP 1212422 B. Additional CTLA-4 antibodies are described in U.S. Pat. Nos. 5,811,097; 5,855,887; 6,051,227; and 6,984,720; in PCT Publication Nos. WO 01/14424 and WO 00/37504; and in U.S. Publication Nos. 2002/0039581 and 2002/086014. Other anti-CTLA-4 antibodies that can be used in a method of the present invention include, for example, those disclosed in: WO 98/42752; U.S. Pat. Nos. 6,682,736 and 6,207,156; Hurwitz et al., Proc. Natl. Acad. Sci. USA, 95(17): 10067-10071 (1998); Camacho et al., J. Clin: Oncology, 22(145): Abstract No. 2505 (2004) (antibody CP-675206); Mokyr et al., Cancer Res., 58:5301-5304 (1998), and U.S. Pat. Nos. 5,977,318, 6,682,736, 7,109,003, and 7,132,281. A preferred clinical CTLA-4 antibody is human monoclonal antibody (also referred to as MDX-010 and Ipilimumab with CAS No. 477202-00-9 and available from Medarex, Inc., Bloomsbury, N.J.) is disclosed in WO 01/14424. With regard to CTLA-4 antagonist (antibodies), these are known and include Tremelimumab (CP-675,206) and Ipilimumab.

In some embodiments, the immunotherapy consists in administering to the patient a combination of a CTLA-4 antagonist and a PD-1 antagonist.

Other immune-checkpoint inhibitors include lymphocyte activation gene-3 (LAG-3) inhibitors, such as IMP321, a soluble Ig fusion protein (Brignone et al., 2007, J. Immunol. 179:4202-4211). Other immune-checkpoint inhibitors include B7 inhibitors, such as B7-H3 and B7-H4 inhibitors. In particular, the anti-B7-H3 antibody MGA271 (Loo et al., 2012, Clin. Cancer Res. July 15 (18) 3834). Also included are TIM-3 (T-cell immunoglobulin domain and mucin domain 3) inhibitors (Fourcade et al., 2010, J. Exp. Med. 207:2175-86 and Sakuishi et al., 2010, J. Exp. Med. 207:2187-94). As used herein, the term “TIM-3” has its general meaning in the art and refers to T cell immunoglobulin and mucin domain-containing molecule 3. The natural ligand of TIM-3 is galectin 9 (Gal9). Accordingly, the term “TIM-3 inhibitor” as used herein refers to a compound, substance or composition that can inhibit the function of TIM-3. For example, the inhibitor can inhibit the expression or activity of TIM-3, modulate or block the TIM-3 signaling pathway and/or block the binding of TIM-3 to galectin-9. Antibodies having specificity for TIM-3 are well known in the art and typically those described in WO2011155607, WO2013006490 and WO2010117057.

In some embodiments, the immune checkpoint inhibitor is an IDO inhibitor. Examples of IDO inhibitors are described in WO 2014150677. Examples of IDO inhibitors include without limitation 1-methyl-tryptophan (IMT), β-(3-benzofuranyl)-alanine, β-(3-benzo(b)thienyl)-alanine), 6-nitro-tryptophan, 6-fluoro-tryptophan, 4-methyl-tryptophan, 5-methyl tryptophan, 6-methyl-tryptophan, 5-methoxy-tryptophan, 5-hydroxy-tryptophan, indole 3-carbinol, 3,3′-diindolylmethane, epigallocatechin gallate, 5-Br-4-Cl-indoxyl 1,3-diacetate, 9-vinylcarbazole, acemetacin, 5-bromo-tryptophan, 5-bromoindoxyl diacetate, 3-Amino-naphtoic acid, pyrrolidine dithiocarbamate, 4-phenylimidazole a brassinin derivative, a thiohydantoin derivative, a β-carboline derivative or a brassilexin derivative. Preferably the IDO inhibitor is selected from 1-methyl-tryptophan, β-(3-benzofuranyl)-alanine, 6-nitro-L-tryptophan, 3-Amino-naphtoic acid and β-[3-benzo(b)thienyl]-alanine or a derivative or prodrug thereof.

As used herein the term “co-administering” as used herein means a process whereby the combination of the agent capable of depleting the population of CD163+ tumor associated macrophages and the immune checkpoint inhibitor, is administered to the same patient. The agent capable of depleting the population of CD163+ tumor associated macrophages and the immune checkpoint inhibitor may be administered simultaneously, at essentially the same time, or sequentially. If administration takes place sequentially, the agent capable of depleting the population of CD163+ tumor associated macrophages is administered before the immune checkpoint inhibitor. The agent capable of depleting the population of CD163+ tumor associated macrophages and the immune checkpoint inhibitor need not be administered by means of the same vehicle. The agent capable of depleting the population of CD163+ tumor associated macrophages and the immune checkpoint inhibitor may be administered one or more times and the number of administrations of each component of the combination may be the same or different. In addition, the agent capable of depleting the population of CD163+ tumor associated macrophages and the immune checkpoint inhibitor need not be administered at the same site.

As used the terms “combination” and “combination therapy” are interchangeable and refer to treatments comprising the administration of at least two compounds administered simultaneously, separately or sequentially. As used herein the term “co-administering” as used herein means a process whereby the combination of at least two compounds is administered to the same patient. The at least two compounds may be administered simultaneously, at essentially the same time, or sequentially. The at least two compounds can be administered separately by means of different vehicles or composition. The at least two compounds can also administered in the same vehicle or composition (e.g. pharmaceutical composition). The at least two compounds may be administered one or more times and the number of administrations of each component of the combination may be the same or different.

In particular, the method of the present invention is particularly suitable for the treatment of cancer characterized by a low tumor infiltration of CD8+ T cells. Accordingly a further object of the present invention relates to a method of treating cancer in a patient in need thereof comprising i) quantifying the density of CD8+ T cells in a tumor tissue sample obtained from the patient ii) comparing the density quantified at step i) with a predetermined reference value and iii) administering to the patient a combine therapeutically effective amount of an agent capable of depleting the population of CD163+ tumor associated macrophages and an immune checkpoint inhibitor when and the density quantified for CD8+ T cells quantified at step i) lower that its corresponding predetermined reference value.

In some embodiments, the method of the present invention is particularly suitable for the treatment of cancer characterized by a low tumor infiltration of CD8+ T cells and a high tumor infiltration of CD163+ tumor associated macrophages. Accordingly a further object of the present invention relates to a method of treating cancer in a patient in need thereof comprising i) quantifying the density of CD8+ T cells and density of CD163+ tumor associated macrophages in a tumor tissue sample obtained from the patient ii) comparing the densities quantified at step i) with their predetermined reference values and iii) administering to the patient a combine therapeutically effective amount of an agent capable of depleting the population of CD163+ tumor associated macrophages and an immune checkpoint inhibitor when the density of CD163+ tumor associated macrophages quantified at step i) is higher than its corresponding predetermined reference value and the density quantified for CD8+ T cells quantified at step i) lower that its corresponding predetermined reference value.

As used herein, the term “tumor tissue sample” means any tissue tumor sample derived from the patient. Said tissue sample is obtained for the purpose of the in vitro evaluation. In some embodiments, the tumor sample may result from the tumor resected from the patient. In some embodiments, the tumor sample may result from a biopsy performed in the primary tumor of the patient or performed in metastatic sample distant from the primary tumor of the patient. For example an endoscopical biopsy performed in the bowel of the patient affected by a colorectal cancer. In some embodiments, the tumor tissue sample encompasses (i) a global primary tumor (as a whole), (ii) a tissue sample from the center of the tumor, (iii) a tissue sample from the tissue directly surrounding the tumor which tissue may be more specifically named the “invasive margin” of the tumor, (iv) lymphoid islets in close proximity with the tumor, (v) the lymph nodes located at the closest proximity of the tumor, (vi) a tumor tissue sample collected prior surgery (for follow-up of patients after treatment for example), and (vii) a distant metastasis. As used herein the “invasive margin” has its general meaning in the art and refers to the cellular environment surrounding the tumor. In some embodiments, the tumor tissue sample, irrespective of whether it is derived from the center of the tumor, from the invasive margin of the tumor, or from the closest lymph nodes, encompasses pieces or slices of tissue that have been removed from the tumor center of from the invasive margin surrounding the tumor, including following a surgical tumor resection or following the collection of a tissue sample for biopsy, for further quantification of one or several biological markers, notably through histology or immunohistochemistry methods, through flow cytometry methods and through methods of gene or protein expression analysis, including genomic and proteomic analysis. The tumor tissue sample can, of course, be patiented to a variety of well-known post-collection preparative and storage techniques (e.g., fixation, storage, freezing, etc.). The sample can be fresh, frozen, fixed (e.g., formalin fixed), or embedded (e.g., paraffin embedded).

In some embodiments, the quantification of density of cells is determined by immunohistochemistry (IHC). For example, the quantification of the density of cells is performed by contacting the tissue tumor tissue sample with a binding partner (e.g. an antibody) specific for a cell surface marker of said cells. Typically, the quantification of density of cells is performed by contacting the tissue tumor tissue sample with a binding partner (e.g. an antibody) specific for CD8 for CD8+ cells and CD163 for CD163+ tumor associated macrophages. Typically, the density of cells is expressed as the number of these cells that are counted per one unit of surface area of tissue sample, e.g. as the number of cells that are counted per cm² or mm² of surface area of tumor tissue sample. In some embodiments, the density of cells may also be expressed as the number of cells per one volume unit of sample, e.g. as the number of cells per cm3 of tumor tissue sample. In some embodiments, the density of cells may also consist of the percentage of the specific cells per total cells (set at 100%). Immunohistochemistry typically includes the following steps i) fixing the tumor tissue sample with formalin, ii) embedding said tumor tissue sample in paraffin, iii) cutting said tumor tissue sample into sections for staining, iv) incubating said sections with the binding partner specific for the marker, v) rinsing said sections, vi) incubating said section with a secondary antibody typically biotinylated and vii) revealing the antigen-antibody complex typically with avidin-biotin-peroxidase complex. Accordingly, the tumor tissue sample is firstly incubated the binding partners. After washing, the labeled antibodies that are bound to marker of interest are revealed by the appropriate technique, depending of the kind of label is borne by the labeled antibody, e.g. radioactive, fluorescent or enzyme label. Multiple labelling can be performed simultaneously. Alternatively, the method of the present invention may use a secondary antibody coupled to an amplification system (to intensify staining signal) and enzymatic molecules. Such coupled secondary antibodies are commercially available, e.g. from Dako, EnVision system. Counterstaining may be used, e.g. H&E, DAPI, Hoechst. Other staining methods may be accomplished using any suitable method or system as would be apparent to one of skill in the art, including automated, semi-automated or manual systems. For example, one or more labels can be attached to the antibody, thereby permitting detection of the target protein (i.e the marker). Exemplary labels include radioactive isotopes, fluorophores, ligands, chemiluminescent agents, enzymes, and combinations thereof. In some embodiments, the label is a quantum dot. Non-limiting examples of labels that can be conjugated to primary and/or secondary affinity ligands include fluorescent dyes or metals (e.g. fluorescein, rhodamine, phycoerythrin, fluorescamine), chromophoric dyes (e.g. rhodopsin), chemiluminescent compounds (e.g. luminal, imidazole) and bioluminescent proteins (e.g. luciferin, luciferase), haptens (e.g. biotin). A variety of other useful fluorescers and chromophores are described in Stryer L (1968) Science 162:526-533 and Brand L and Gohlke J R (1972) Annu. Rev. Biochem. 41:843-868. Affinity ligands can also be labeled with enzymes (e.g. horseradish peroxidase, alkaline phosphatase, beta-lactamase), radioisotopes (e.g. ³H, ¹⁴C, ³²P, ³⁵S or ¹²⁵I) and particles (e.g. gold). The different types of labels can be conjugated to an affinity ligand using various chemistries, e.g. the amine reaction or the thiol reaction. However, other reactive groups than amines and thiols can be used, e.g. aldehydes, carboxylic acids and glutamine. Various enzymatic staining methods are known in the art for detecting a protein of interest. For example, enzymatic interactions can be visualized using different enzymes such as peroxidase, alkaline phosphatase, or different chromogens such as DAB, AEC or Fast Red. In other examples, the antibody can be conjugated to peptides or proteins that can be detected via a labeled binding partner or antibody. In an indirect IHC assay, a secondary antibody or second binding partner is necessary to detect the binding of the first binding partner, as it is not labeled. The resulting stained specimens are each imaged using a system for viewing the detectable signal and acquiring an image, such as a digital image of the staining. Methods for image acquisition are well known to one of skill in the art. For example, once the sample has been stained, any optical or non-optical imaging device can be used to detect the stain or biomarker label, such as, for example, upright or inverted optical microscopes, scanning confocal microscopes, cameras, scanning or tunneling electron microscopes, canning probe microscopes and imaging infrared detectors. In some examples, the image can be captured digitally. The obtained images can then be used for quantitatively or semi-quantitatively determining the amount of the marker in the sample. Various automated sample processing, scanning and analysis systems suitable for use with immunohistochemistry are available in the art. Such systems can include automated staining and microscopic scanning, computerized image analysis, serial section comparison (to control for variation in the orientation and size of a sample), digital report generation, and archiving and tracking of samples (such as slides on which tissue sections are placed). Cellular imaging systems are commercially available that combine conventional light microscopes with digital image processing systems to perform quantitative analysis on cells and tissues, including immunostained samples. See, e.g., the CAS-200 system (Becton, Dickinson & Co.). In particular, detection can be made manually or by image processing techniques involving computer processors and software. Using such software, for example, the images can be configured, calibrated, standardized and/or validated based on factors including, for example, stain quality or stain intensity, using procedures known to one of skill in the art (see e.g., published U.S. Patent Publication No. US20100136549). The image can be quantitatively or semi-quantitatively analyzed and scored based on staining intensity of the sample. Quantitative or semi-quantitative histochemistry refers to method of scanning and scoring samples that have undergone histochemistry, to identify and quantitate the presence of the specified biomarker (i.e. the marker). Quantitative or semi-quantitative methods can employ imaging software to detect staining densities or amount of staining or methods of detecting staining by the human eye, where a trained operator ranks results numerically. For example, images can be quantitatively analyzed using a pixel count algorithms (e.g., Aperio Spectrum Software, Automated QUantitatative Analysis platform (AQUA® platform), and other standard methods that measure or quantitate or semi-quantitate the degree of staining; see e.g., U.S. Pat. Nos. 8,023,714; 7,257,268; 7,219,016; 7,646,905; published U.S. Patent Publication No. US20100136549 and 20110111435; Camp et al. (2002) Nature Medicine, 8:1323-1327; Bacus et al. (1997) Analyt Quant Cytol Histol, 19:316-328). A ratio of strong positive stain (such as brown stain) to the sum of total stained area can be calculated and scored. The amount of the detected biomarker (i.e. the marker) is quantified and given as a percentage of positive pixels and/or a score. For example, the amount can be quantified as a percentage of positive pixels. In some examples, the amount is quantified as the percentage of area stained, e.g., the percentage of positive pixels. For example, a sample can have at least or about at least or about 0, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 40%, 45%, 50%, 55%, 60%, 65%, 70%, 75%, 80%, 85%, 90%, 95% or more positive pixels as compared to the total staining area. In some embodiments, a score is given to the sample that is a numerical representation of the intensity or amount of the histochemical staining of the sample, and represents the amount of target biomarker (e.g., the marker) present in the sample. Optical density or percentage area values can be given a scaled score, for example on an integer scale. Thus, in some embodiments, the method of the present invention comprises the steps consisting in i) providing one or more immunostained slices of tissue section obtained by an automated slide-staining system by using a binding partner capable of selectively interacting with the marker (e.g. an antibody as above described), ii) proceeding to digitalisation of the slides of step a. by high resolution scan capture, iii) detecting the slice of tissue section on the digital picture iv) providing a size reference grid with uniformly distributed units having a same surface, said grid being adapted to the size of the tissue section to be analyzed, and v) detecting, quantifying and measuring intensity of stained cells in each unit whereby the number or the density of cells stained of each unit is assessed.

In some embodiments, the predetermined value is a threshold value or a cut-off value. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. For example, retrospective measurement of cell densities in properly banked historical patient samples may be used in establishing the predetermined reference value. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. For example, after quantifying the density of cells in a group of reference, one can use algorithmic analysis for the statistic treatment of the measured densities in samples to be tested, and thus obtain a classification standard having significance for sample classification. The full name of ROC curve is receiver operator characteristic curve, which is also known as receiver operation characteristic curve. It is mainly used for clinical biochemical diagnostic tests. ROC curve is a comprehensive indicator that reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is quite high. This algorithmic method is preferably done with a computer. Existing software or systems in the art may be used for the drawing of the ROC curve, such as: MedCalc 9.2.0.1 medical statistical software, SPSS 9.0, ROCPOWER.SAS, DESIGNROC.FOR, MULTIREADER POWER.SAS, CREATE-ROC.SAS, GB STAT VI0.0 (Dynamic Microsystems, Inc. Silver Spring, Md., USA), etc. In some embodiments, the predetermined reference value correlates with the survival time of the patient. Those of skill in the art will recognize that OS survival time is generally based on and expressed as the percentage of people who survive a certain type of cancer for a specific amount of time. Cancer statistics often use an overall five-year survival rate. In general, OS rates do not specify whether cancer survivors are still undergoing treatment at five years or if they've become cancer-free (achieved remission). DSF gives more specific information and is the number of people with a particular cancer who achieve remission. Also, progression-free survival (PFS) rates (the number of people who still have cancer, but their disease does not progress) includes people who may have had some success with treatment, but the cancer has not disappeared completely. As used herein, the expression “short survival time” indicates that the patient will have a survival time that will be lower than the median (or mean) observed in the general population of patients suffering from said cancer. When the patient will have a short survival time, it is meant that the patient will have a “poor prognosis”. Inversely, the expression “long survival time” indicates that the patient will have a survival time that will be higher than the median (or mean) observed in the general population of patients suffering from said cancer. When the patient will have a long survival time, it is meant that the patient will have a “good prognosis”.

A further object of the present invention relates to a method of treating a cancer resistant to immune checkpoint therapy in a subject in need thereof comprising administering to the subject a therapeutically effective amount of an agent capable of depleting the population of CD163+ tumor associated macrophages.

As used herein the term “resistance to immune checkpoint therapy” is used in its broadest context to refer to the reduced effectiveness of at least one immune checkpoint inhibitor (e.g. PD-1 antagonist) to inhibit the growth of a cell, kill a cell or inhibit one or more cellular functions, and to the ability of a cell to survive exposure to an agent designed to inhibit the growth of the cell, kill the cell or inhibit one or more cellular functions. The resistance displayed by a cell may be acquired, for example by prior exposure to the agent, or may be inherent or innate. The resistance displayed by a cell may be complete in that the agent is rendered completely ineffective against the cell, or may be partial in that the effectiveness of the agent is reduced. Accordingly, the term “resistant” refers to the repeated outbreak of cancer, or a progression of cancer independently of whether the disease was cured before said outbreak or progression.

A further object of the present invention relates to a method for enhancing the potency/efficacy of an immune checkpoint inhibitor administered to a subject suffering from a cancer as part of a treatment regimen, the method comprising administering to the subject a pharmaceutically effective amount of an agent capable of depleting the population of CD163+ tumor associated macrophages in combination with at least one immune checkpoint inhibitor.

As used herein, the expression “enhancing the potency of an immune checkpoint” refers to the ability of the agent capable of depleting the population of CD163+ tumor associated macrophages to increase the ability of the immune checkpoint inhibitor to enhance the proliferation, migration, persistence and/or cytoxic activity of CD8+ T cells. The ability of the immune checkpoint inhibitor to enhance T CD8 cell killing activity may be determined by any assay well known in the art. Typically said assay is an in vitro assay wherein CD8+ T cells are brought into contact with target cells (e.g. target cells that are recognized and/or lysed by CD8+ T cells). For example, the immune checkpoint inhibitor of the present invention can be selected for the ability to increase specific lysis by CD8+ T cells by more than about 20%, preferably with at least about 30%, at least about 40%, at least about 50%, or more of the specific lysis obtained at the same effector: target cell ratio with CD8+ T cells or CD8 T cell lines that are contacted by the immune checkpoint inhibitor of the present invention, Examples of protocols for classical cytotoxicity assays are conventional.

As used herein, the expression “enhanced therapeutic efficacy” relative to cancer refers to a slowing or diminution of the growth of cancer cells or a solid tumor, or a reduction in the total number of cancer cells or total tumor burden. An “improved therapeutic outcome” or “enhanced therapeutic efficacy” therefore means there is an improvement in the condition of the patient according to any clinically acceptable criteria, including, for example, decreased tumor size, an increase in time to tumor progression, increased progression-free survival, increased overall survival time, an increase in life expectancy, or an improvement in quality of life. In particular, “improved” or “enhanced” refers to an improvement or enhancement of 1%, 5%, 10%, 25%, 50%, 75%, 100%, or greater than 100% of any clinically acceptable indicator of therapeutic outcome or efficacy. As used herein, the expression “relative to” when used in the context of comparing the activity and/or efficacy of a combination composition comprising the immune checkpoint inhibitor with the agent capable of depleting the population of CD163+ tumor associated macrophages to the activity and/or efficacy of the immune checkpoint inhibitor alone, refers to a comparison using amounts known to be comparable according to one of skill in the art.

A further object of the present invention relates to a method of preventing resistance to an administered immune checkpoint inhibitor in a subject suffering from a cancer comprising administering to the subject a therapeutically effective amount of an agent capable of depleting the population of CD163+ tumor associated macrophages.

As used herein, the term “agent capable of depleting the population of CD163+ tumor associated macrophages” refers to any compound that is able to deplete said populations. As used herein, the term “deplete” with respect to CD163+ tumor associated macrophages, refers to a measurable decrease in the number of CD163+ TAM in the subject's tumor. The reduction can be at least about 10%, e.g., at least about 20%, 30%, 40%, 50%, 60%, 70%, 80%, 90%, 95%, 96%, 97%, 98%, 99%, or more. In some embodiments, the term refers to a decrease in the number of CD163+ TAM in a subject's tumor to an amount below detectable limits.

In some embodiments, the agent is an antibody having binding affinity for CD163 and that leads to the depletion of CD163+ TAMs in the subject's tumor. In particular, the antibody binds to the extracellular domain of CD163 as defined above.

As used herein, the term “antibody” is thus used to refer to any antibody-like molecule that has an antigen binding region, and this term includes antibody fragments that comprise an antigen binding domain such as Fab′, Fab, F(ab′)2, single domain antibodies (DABs), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa(lamda) bodies (scFv-CL fusions); BiTE (Bispecific T-cell Engager, scFv-scFv tandems to attract T cells); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics comprising one or more CDRs and the like. The techniques for preparing and using various antibody-based constructs and fragments are well known in the art (see Kabat et al., 1991, specifically incorporated herein by reference). Diabodies, in particular, are further described in EP 404, 097 and WO 93/1 1 161; whereas linear antibodies are further described in Zapata et al. (1995). Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, Fv, dsFv, Fd, dAbs, TandAbs, ds-scFv, dimers, minibodies, diabodies, bispecific antibody fragments and other fragments can also be synthesized by recombinant techniques or can be chemically synthesized. Techniques for producing antibody fragments are well known and described in the art. For example, each of Beckman et al., 2006; Holliger & Hudson, 2005; Le Gall et al., 2004; Reff & Heard, 2001; Reiter et al., 1996; and Young et al., 1995 further describe and enable the production of effective antibody fragments. In some embodiments, the antibody of the present invention is a single chain antibody. As used herein the term “single domain antibody” has its general meaning in the art and refers to the single heavy chain variable domain of antibodies of the type that can be found in Camelid mammals which are naturally devoid of light chains. Such single domain antibody are also “Nanobody®”. For a general description of (single) domain antibodies, reference is also made to the prior art cited above, as well as to EP 0 368 684, Ward et al. (Nature 1989 Oct. 12; 341 (6242): 544-6), Holt et al., Trends Biotechnol., 2003, 21(11):484-490; and WO 06/030220, WO 06/003388.

As used herein the term “bind” indicates that the antibody has affinity for the surface molecule. The term “affinity”, as used herein, means the strength of the binding of an antibody to an epitope. The affinity of an antibody is given by the dissociation constant Kd, defined as [Ab]×[Ag]/[Ab-Ag], where [Ab-Ag] is the molar concentration of the antibody-antigen complex, [Ab] is the molar concentration of the unbound antibody and [Ag] is the molar concentration of the unbound antigen. The affinity constant Ka is defined by 1/Kd. Preferred methods for determining the affinity of mAbs can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference. One preferred and standard method well known in the art for determining the affinity of mAbs is the use of Biacore instruments.

In natural antibodies, two heavy chains are linked to each other by disulfide bonds and each heavy chain is linked to a light chain by a disulfide bond. There are two types of light chain, lambda (l) and kappa (κ). There are five main heavy chain classes (or isotypes) which determine the functional activity of an antibody molecule: IgM, IgD, IgG, IgA and IgE. Each chain contains distinct sequence domains. The light chain includes two domains, a variable domain (VL) and a constant domain (CL). The heavy chain includes four domains, a variable domain (VH) and three constant domains (CHI, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from nonhypervariable or framework regions (FR) can participate to the antibody binding site or influence the overall domain structure and hence the combining site. Complementarity Determining Regions or CDRs refer to amino acid sequences which together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, typically includes six CDRs, comprising the CDR set from each of a heavy and a light chain V region. Framework Regions (FRs) refer to amino acid sequences interposed between CDRs. The residues in antibody variable domains are conventionally numbered according to a system devised by Kabat et al. This system is set forth in Kabat et al., 1987, in Sequences of Proteins of Immunological Interest, US Department of Health and Human Services, NIH, USA (hereafter “Kabat et al.”). This numbering system is used in the present specification. The Kabat residue designations do not always correspond directly with the linear numbering of the amino acid residues in SEQ ID sequences. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence. The CDRs of the heavy chain variable domain are located at residues 31-35B (H-CDR1), residues 50-65 (H-CDR2) and residues 95-102 (H-CDR3) according to the Kabat numbering system. The CDRs of the light chain variable domain are located at residues 24-34 (L-CDR1), residues 50-56 (L-CDR2) and residues 89-97 (L-CDR3) according to the Kabat numbering system.

In some embodiments, the antibody is a humanized antibody. As used herein, “humanized” describes antibodies wherein some, most or all of the amino acids outside the CDR regions are replaced with corresponding amino acids derived from human immunoglobulin molecules. Methods of humanization include, but are not limited to, those described in U.S. Pat. Nos. 4,816,567, 5,225,539, 5,585,089, 5,693,761, 5,693,762 and 5,859,205, which are hereby incorporated by reference.

In some embodiments, the antibody is a fully human antibody. Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference.

In some embodiments, the antibody suitable for depletion of CD163+ TAM mediates antibody-dependent cell-mediated cytotoxicity. As used herein the term “antibody-dependent cell-mediated cytotoxicity” or ‘ADCC” refer to a cell-mediated reaction in which non-specific cytotoxic cells (e.g., Natural Killer (NK) cells, neutrophils, and macrophages) recognize bound antibody on a target cell and subsequently cause lysis of the target cell. While not wishing to be limited to any particular mechanism of action, these cytotoxic cells that mediate ADCC generally express Fc receptors (FcRs).

As used herein “Fc region” includes the polypeptides comprising the constant region of an antibody excluding the first constant region immunoglobulin domain. Thus Fc refers to the last two constant region immunoglobulin domains of IgA, IgD, and IgG, and the last three constant region immunoglobulin domains of IgE and IgM, and the flexible hinge N-terminal to these domains. For IgA and IgM Fc may include the J chain. For IgG, Fc comprises immunoglobulin domains Cgamma2 and Cgamma3 (Cy2 and Cy3) and the hinge between Cgamma1 (Cγ1) and Cgamma2 (Cy2). Although the boundaries of the Fc region may vary, the human IgG heavy chain Fc region is usually defined to comprise residues C226 or P230 to its carboxyl-terminus, wherein the numbering is according to the EU index as in Kabat et al. (1991, NIH Publication 91-3242, National Technical Information Service, Springfield, Va.). The “EU index as set forth in Kabat” refers to the residue numbering of the human IgG1 EU antibody as described in Kabat et al. supra. Fc may refer to this region in isolation, or this region in the context of an antibody, antibody fragment, or Fc fusion protein. An Fc variant protein may be an antibody, Fe fusion, or any protein or protein domain that comprises an Fc region. Particularly preferred are proteins comprising variant Fe regions, which are non-naturally occurring variants of an Fc region. The amino acid sequence of a non-naturally occurring Fe region (also referred to herein as a “variant Fc region”) comprises a substitution, insertion and/or deletion of at least one amino acid residue compared to the wild type amino acid sequence. Any new amino acid residue appearing in the sequence of a variant Fc region as a result of an insertion or substitution may be referred to as a non-naturally occurring amino acid residue. Note: Polymorphisms have been observed at a number of Fc positions, including but not limited to Kabat 270, 272, 312, 315, 356, and 358, and thus slight differences between the presented sequence and sequences in the prior art may exist.

The terms “Fe receptor” or “FcR” are used to describe a receptor that binds to the Fc region of an antibody. The primary cells for mediating ADCC, NK cells, express FcγRIII, whereas monocytes express FcγRI, FcγRII, FcγRIII and/or FcγRIV. FcR expression on hematopoietic cells is summarized in Ravetch and Kinet, Annu. Rev. Immunol., 9:457-92 (1991). To assess ADCC activity of a molecule, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecules of interest may be assessed in vivo, e.g., in an animal model such as that disclosed in Clynes et al., Proc. Natl. Acad. Sci. (USA), 95:652-656 (1998). As used herein, the term Effector cells” are leukocytes which express one or more FcRs and perform effector functions. The cells express at least FcγRI, FCγRII, FcγRIII and/or FcγRIV and carry out ADCC effector function. Examples of human leukocytes which mediate ADCC include peripheral blood mononuclear cells (PBMC), natural killer (NK) cells, monocytes, cytotoxic T cells and neutrophils.

In some embodiments, the antibody suitable for depletion of CD163+ TAM is a full-length antibody. In some embodiments, the full-length antibody is an IgG1 antibody. In some embodiments, the full-length antibody is an IgG3 antibody.

In some embodiments, the antibody suitable for depletion of CD163+ TAM comprises a variant Fe region that has an increased affinity for FcγRIA, FcγRIIA, FcγRIIB, FcγRIIIA, FcγRIIIB, and FcγRIV. In some embodiments, the antibody of the present invention comprises a variant Fe region comprising at least one amino acid substitution, insertion or deletion wherein said at least one amino acid residue substitution, insertion or deletion results in an increased affinity for FcγRIA, FcγRIIA, FcγRIIB, FcγRIIIA, FcγRIIIB, and FcγRIV, In some embodiments, the antibody of the present invention comprises a variant Fc region comprising at least one amino acid substitution, insertion or deletion wherein said at least one amino acid residue is selected from the group consisting of: residue 239, 330, and 332, wherein amino acid residues are numbered following the EU index. In some embodiments, the antibody of the present invention comprises a variant Fc region comprising at least one amino acid substitution wherein said at least one amino acid substitution is selected from the group consisting of: S239D, A330L, A330Y, and 1332E, wherein amino acid residues are numbered following the EU index.

In some embodiments, the glycosylation of the antibody suitable for depletion of CD163+ TAM is modified. For example, an aglycosylated antibody can be made (i.e., the antibody lacks glycosylation). Glycosylation can be altered to, for example, increase the affinity of the antibody for the antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 by Co et al. Additionally or alternatively, an antibody can be made that has an altered type of glycosylation, such as a hypofucosylated or non-fucosylated antibody having reduced amounts of or no fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the present invention to thereby produce an antibody with altered glycosylation. For example, EP 1,176,195 by Hang et al. describes a cell line with a functionally disrupted FUT8 gene, which encodes a fucosyl transferase, such that antibodies expressed in such a cell line exhibit hypofucosylation or are devoid of fucosyl residues. Therefore, in some embodiments, the human monoclonal antibodies of the present invention may be produced by recombinant expression in a cell line which exhibit hypofucosylation or non-fucosylation pattern, for example, a mammalian cell line with deficient expression of the FUT8 gene encoding fucosyltransferase. PCT Publication WO 03/035835 by Presta describes a variant CHO cell line, Lecl3 cells, with reduced ability to attach fucose to Asn(297)-linked carbohydrates, also resulting in hypofucosylation of antibodies expressed in that host cell (see also Shields, R. L. et al, 2002 J. Biol. Chem. 277:26733-26740). PCT Publication WO 99/54342 by Umana et al. describes cell lines engineered to express glycoprotein-modifying glycosyl transferases (e.g., beta(1,4)-N acetylglucosaminyltransferase III (GnTIII)) such that antibodies expressed in the engineered cell lines exhibit increased bisecting GlcNac structures which results in increased ADCC activity of the antibodies (see also Umana et al, 1999 Nat. Biotech. 17: 176-180). Eureka Therapeutics further describes genetically engineered CHO mammalian cells capable of producing antibodies with altered mammalian glycosylation pattern devoid of fucosyl residues (http://www.eurekainc.com/a&boutus/companyoverview.html). Alternatively, the human monoclonal antibodies of the present invention can be produced in yeasts or filamentous fungi engineered for mammalian-like glycosylation pattern and capable of producing antibodies lacking fucose as glycosylation pattern (see for example EP1297172B1 In some embodiments, the antibody suitable for depletion of CD163+ TAM mediates complement dependent cytotoxicity. “Complement dependent cytotoxicity” or “CDC” refers to the ability of a molecule to initiate complement activation and lyse a target in the presence of complement. The complement activation pathway is initiated by the binding of the first component of the complement system (C1q) to a molecule (e.g., an antibody) complexed with a cognate antigen. To assess complement activation, a CDC assay, e.g., as described in Gazzano-Santaro et al., J. Immunol. Methods, 202:163 (1996), may be performed.

In some embodiments, the antibody suitable for depletion of CD163+ TAM mediates antibody-dependent phagocytosis. As used herein, the term “antibody-dependent phagocytosis” or “opsonisation” refers to the cell-mediated reaction wherein nonspecific cytotoxic cells that express FcγRs recognize bound antibody on a target cell and subsequently cause phagocytosis of the target cell.

In some embodiments, the antibody suitable for depletion of CD163+ TAM is a multispecific antibody comprising a first antigen binding site directed against CD163 and at least one second antigen binding site directed against an effector cell as above described. In said embodiments, the second antigen-binding site is used for recruiting a killing mechanism such as, for example, by binding an antigen on a human effector cell. In some embodiments, an effector cell is capable of inducing ADCC, such as a natural killer cell. For example, monocytes, macrophages, which express FcRs, are involved in specific killing of target cells and presenting antigens to other components of the immune system. In some embodiments, an effector cell may phagocytose a target antigen or target cell. The expression of a particular FcR on an effector cell may be regulated by humoral factors such as cytokines. An effector cell can phagocytose a target antigen or phagocytose or lyse a target cell. Suitable cytotoxic agents and second therapeutic agents are exemplified below, and include toxins (such as radiolabeled peptides), chemotherapeutic agents and prodrugs. In some embodiments, the second binding site binds to a Fc receptor as above defined. In some embodiments, the second binding site binds to a surface molecule of NK cells so that said cells can be activated. In some embodiments, the second binding site binds to NKp46. Exemplary formats for the multispecific antibody molecules of the present invention include, but are not limited to (i) two antibodies cross-linked by chemical heteroconjugation, one with a specificity to a specific surface molecule of ILC and another with a specificity to a second antigen; (ii) a single antibody that comprises two different antigen-binding regions; (iii) a single-chain antibody that comprises two different antigen-binding regions, e.g., two scFvs linked in tandem by an extra peptide linker; (iv) a dual-variable-domain antibody (DVD-Ig), where each light chain and heavy chain contains two variable domains in tandem through a short peptide linkage (Wu et al., Generation and Characterization of a Dual Variable Domain Immunoglobulin (DVD-Ig™) Molecule, In: Antibody Engineering, Springer Berlin Heidelberg (2010)); (v) a chemically-linked bispecific (Fab′)2 fragment; (vi) a Tandab, which is a fusion of two single chain diabodies resulting in a tetravalent bispecific antibody that has two binding sites for each of the target antigens; (vii) a flexibody, which is a combination of scFvs with a diabody resulting in a multivalent molecule; (viii) a so called “dock and lock” molecule, based on the “dimerization and docking domain” in Protein Kinase A, which, when applied to Fabs, can yield a trivalent bispecific binding protein consisting of two identical Fab fragments linked to a different Fab fragment; (ix) a so-called Scorpion molecule, comprising, e.g., two scFvs fused to both termini of a human Fab-arm; and (x) a diabody. Another exemplary format for bispecific antibodies is IgG-like molecules with complementary CH3 domains to force heterodimerization. Such molecules can be prepared using known technologies, such as, e.g., those known as Triomab/Quadroma (Trion Pharma/Fresenius Biotech), Knob-into-Hole (Genentech), CrossMAb (Roche) and electrostatically-matched (Amgen), LUZ-Y (Genentech), Strand Exchange Engineered Domain body (SEEDbody)(EMD Serono), Biclonic (Merus) and DuoBody (Genmab A/S) technologies.

In some embodiments, the antibody suitable for depletion of CD163+ TAM is conjugated to a therapeutic moiety, i.e. a drug. The therapeutic moiety can be, e.g., a cytotoxin, a chemotherapeutic agent, a cytokine, an immunosuppressant, an immune stimulator, a lytic peptide, or a radioisotope. Such conjugates are referred to herein as an “antibody-drug conjugates” or “ADCs”.

In some embodiments, the antibody suitable for depletion of CD163+ TAM is conjugated to a cytotoxic moiety. The cytotoxic moiety may, for example, be selected from the group consisting of taxol; cytochalasin B; gramicidin D; ethidium bromide; emetine; mitomycin; etoposide; tenoposide; vincristine; vinblastine; colchicin; doxorubicin; daunorubicin; dihydroxy anthracin dione; a tubulin-inhibitor such as maytansine or an analog or derivative thereof; an antimitotic agent such as monomethyl auristatin E or F or an analog or derivative thereof; dolastatin 10 or 15 or an analogue thereof; irinotecan or an analogue thereof; mitoxantrone; mithramycin; actinomycin D; 1-dehydrotestosterone; a glucocorticoid; procaine; tetracaine; lidocaine; propranolol; puromycin; calicheamicin or an analog or derivative thereof; an antimetabolite such as methotrexate, 6 mercaptopurine, 6 thioguanine, cytarabine, fludarabin, 5 fluorouracil, decarbazine, hydroxyurea, asparaginase, gemcitabine, or cladribine; an alkylating agent such as mechlorethamine, thioepa, chlorambucil, melphalan, carmustine (BSNU), lomustine (CCNU), cyclophosphamide, busulfan, dibromomannitol, streptozotocin, dacarbazine (DTIC), procarbazine, mitomycin C; a platinum derivative such as cisplatin or carboplatin; duocarmycin A, duocarmycin SA, rachelmycin (CC-1065), or an analog or derivative thereof; an antibiotic such as dactinomycin, bleomycin, daunorubicin, doxorubicin, idarubicin, mithramycin, mitomycin, mitoxantrone, plicamycin, anthramycin (AMC)); pyrrolo[2,1-c][1,4]-benzodiazepines (PDB); diphtheria toxin and related molecules such as diphtheria A chain and active fragments thereof and hybrid molecules, ricin toxin such as ricin A or a deglycosylated ricin A chain toxin, cholera toxin, a Shiga-like toxin such as SLT I, SLT II, SLT IIV, LT toxin, C3 toxin, Shiga toxin, pertussis toxin, tetanus toxin, soybean Bowman-Birk protease inhibitor, Pseudomonas exotoxin, alorin, saporin, modeccin, gelanin, abrin A chain, modeccin A chain, alpha-sarcin, Aleurites fordii proteins, dianthin proteins, Phytolacca americana proteins such as PAPI, PAPII, and PAP-S, Momordica charantia inhibitor, curcin, crotin, Sapaonaria officinalis inhibitor, gelonin, mitogellin, restrictocin, phenomycin, and enomycin toxins; ribonuclease (RNase); DNase I, Staphylococcal enterotoxin A; pokeweed antiviral protein; diphtherin toxin; and Pseudomonas endotoxin.

In some embodiments, the antibody suitable for depletion of CD163+ TAM is conjugated to an auristatin or a peptide analog, derivative or prodrug thereof. Auristatins have been shown to interfere with microtubule dynamics, GTP hydrolysis and nuclear and cellular division (Woyke et al (2001) Antimicrob. Agents and Chemother. 45(12): 3580-3584) and have anti-cancer (U.S. Pat. No. 5,663,149) and antifungal activity (Pettit et al., (1998) Antimicrob. Agents and Chemother. 42: 2961-2965. For example, auristatin E can be reacted with para-acetyl benzoic acid or benzoylvaleric acid to produce AEB and AEVB, respectively. Other typical auristatin derivatives include AFP, MMAF (monomethyl auristatin F), and MMAE (monomethyl auristatin E). Suitable auristatins and auristatin analogs, derivatives and prodrugs, as well as suitable linkers for conjugation of auristatins to Abs, are described in, e.g., U.S. Pat. Nos. 5,635,483, 5,780,588 and 6,214,345 and in International patent application publications WO02088172, WO2004010957, WO2005081711, WO2005084390, WO2006132670, WO03026577, WO200700860, WO207011968 and WO205082023.

In some embodiments, the antibody suitable for depletion of CD163+ TAM is conjugated to pyrrolo[2,1-c][1,4]-benzodiazepine (PDB) or an analog, derivative or prodrug thereof. Suitable PDBs and PDB derivatives, and related technologies are described in, e.g., Hartley J. A. et al., Cancer Res 2010; 70(17): 6849-6858; Antonow D. et al., Cancer J 2008; 14(3): 154-169; Howard P. W. et al., Bioorg Med Chem Lett 2009; 19: 6463-6466 and Sagnou et al., Bioorg Med Chem Lett 2000; 10(18): 2083-2086. In some embodiments, the antibody is conjugated to pyrrolobenzodiazepine (PBD) as typically described in WO2017059289.

In some embodiments, the antibody suitable for depletion of CD163+ TAM is conjugated to a cytotoxic moiety selected from the group consisting of an anthracycline, maytansine, calicheamicin, duocarmycin, rachelmycin (CC-1065), dolastatin 10, dolastatin 15, irinotecan, monomethyl auristatin E, monomethyl auristatin F, a PDB, or an analog, derivative, or prodrug of any thereof.

In some embodiments, the antibody suitable for depletion of CD163+ TAM is conjugated to an anthracycline or an analog, derivative or prodrug thereof. In some embodiments, the antibody is conjugated to maytansine or an analog, derivative or prodrug thereof. In some embodiments, the antibody is conjugated to calicheamicin or an analog, derivative or prodrug thereof. In some embodiments, the antibody is conjugated to duocarmycin or an analog, derivative or prodrug thereof. In some embodiments, the antibody is conjugated to rachelmycin (CC-1065) or an analog, derivative or prodrug thereof. In some embodiments, the antibody is conjugated to dolastatin 10 or an analog, derivative or prodrug thereof. In some embodiments, the antibody is conjugated to dolastatin 15 or an analog, derivative or prodrug thereof. In some embodiments, the antibody is conjugated to monomethyl auristatin E or an analog, derivative or prodrug thereof. In some embodiments, the antibody is conjugated to monomethyl auristatin F or an analog, derivative or prodrug thereof. In some embodiments, the antibody is conjugated to pyrrolo[2,1-c][1,4]-benzodiazepine or an analog, derivative or prodrug thereof. In some embodiments, the antibody is conjugated to irinotecan or an analog, derivative or prodrug thereof.

In some embodiments, the antibody suitable for depletion of CD163+ TAM is conjugated to a nucleic acid or nucleic acid-associated molecule. In one such embodiment, the conjugated nucleic acid is a cytotoxic ribonuclease (RNase) or deoxy-ribonuclease (e.g., DNase I), an antisense nucleic acid, an inhibitory RNA molecule (e.g., a siRNA molecule) or an immunostimulatory nucleic acid (e.g., an immunostimulatory CpG motif-containing DNA molecule). In some embodiments, the antibody is conjugated to an aptamer or a ribozyme.

Techniques for conjugating molecule to antibodies, are well-known in the art (See, e.g., Arnon et al., “Monoclonal Antibodies For Immunotargeting Of Drugs In Cancer Therapy,” in Monoclonal Antibodies And Cancer Therapy (Reisfeld et al. eds., Alan R. Liss, Inc., 1985); Hellstrom et al., “Antibodies For Drug Delivery,” in Controlled Drug Delivery (Robinson et al. eds., Marcel Deiker, Inc., 2nd ed. 1987); Thorpe, “Antibody Carriers Of Cytotoxic Agents In Cancer Therapy: A Review,” in Monoclonal Antibodies '84: Biological And Clinical Applications (Pinchera et al. eds., 1985); “Analysis, Results, and Future Prospective of the Therapeutic Use of Radiolabeled Antibody In Cancer Therapy,” in Monoclonal Antibodies For Cancer Detection And Therapy (Baldwin et al. eds., Academic Press, 1985); and Thorpe et al., 1982, Immunol. Rev. 62:119-58. See also, e.g., PCT publication WO 89/12624.) Typically, the nucleic acid molecule is covalently attached to lysines or cysteines on the antibody, through N-hydroxysuccinimide ester or maleimide functionality respectively. Methods of conjugation using engineered cysteines or incorporation of unnatural amino acids have been reported to improve the homogeneity of the conjugate (Axup, J. Y., Bajjuri, K. M., Ritland, M., Hutchins, B. M., Kim, C. H., Kazane, S. A., Halder, R., Forsyth, J. S., Santidrian, A. F., Stafin, K., et al. (2012). Synthesis of site-specific antibody-drug conjugates using unnatural amino acids. Proc. Natl. Acad. Sci. USA 109, 16101-16106.; Junutula, J. R., Flagella, K. M., Graham, R. A., Parsons, K. L., Ha, E., Raab, H., Bhakta, S., Nguyen, T., Dugger, D. L., Li, G., et al. (2010). Engineered thio-trastuzumab-DM1 conjugate with an improved therapeutic index to target humanepidermal growth factor receptor 2-positive breast cancer. Clin. Cancer Res. 16, 4769-4778.). Junutula et al. (2008) developed cysteine-based site-specific conjugation called “THIOMABs” (TDCs) that are claimed to display an improved therapeutic index as compared to conventional conjugation methods. Conjugation to unnatural amino acids that have been incorporated into the antibody is also being explored for ADCs; however, the generality of this approach is yet to be established (Axup et al., 2012). In particular the one skilled in the art can also envisage Fc-containing polypeptide engineered with an acyl donor glutamine-containing tag (e.g., Gin-containing peptide tags or Q-tags) or an endogenous glutamine that are made reactive by polypeptide engineering (e.g., via amino acid deletion, insertion, substitution, or mutation on the polypeptide). Then a transglutaminase, can covalently crosslink with an amine donor agent (e.g., a small molecule comprising or attached to a reactive amine) to form a stable and homogenous population of an engineered Fc-containing polypeptide conjugate with the amine donor agent being site-specifically conjugated to the Fc-containing polypeptide through the acyl donor glutamine-containing tag or the accessible/exposed/reactive endogenous glutamine (WO 2012059882).

Typically the agent capable of depleting the population of CD163+ tumor associated macrophages and the immune checkpoint inhibitor are administered to the patient in a therapeutically effective amount. As used herein, the term “therapeutically effective amount” refers to an amount effective, at dosages and for periods of time necessary, to achieve a desired therapeutic result. A therapeutically effective amount of the active agent may vary according to factors such as the disease state, age, sex, and weight of the individual, and the ability of the active agent to elicit a desired response in the individual. A therapeutically effective amount is also one in which any toxic or detrimental effects of the antibody or antibody portion are outweighed by the therapeutically beneficial effects. The efficient dosages and dosage regimens for the active agent depend on the disease or condition to be treated and may be determined by the persons skilled in the art. A physician having ordinary skill in the art may readily determine and prescribe the effective amount of the pharmaceutical composition required. For example, the physician could start doses of active agent employed in the pharmaceutical composition at levels lower than that required achieving the desired therapeutic effect and gradually increasing the dosage until the desired effect is achieved. In general, a suitable dose of a composition of the present invention will be that amount of the compound, which is the lowest dose effective to produce a therapeutic effect according to a particular dosage regimen. Such an effective dose will generally depend upon the factors described above. For example, a therapeutically effective amount for therapeutic use may be measured by its ability to stabilize the progression of disease. Typically, the ability of a compound to inhibit cancer may, for example, be evaluated in an animal model system predictive of efficacy in human tumors. A therapeutically effective amount of a therapeutic compound may decrease tumor size, or otherwise ameliorate symptoms in a patient. One of ordinary skill in the art would be able to determine such amounts based on such factors as the patient's size, the severity of the patient's symptoms, and the particular composition or route of administration selected. An exemplary, non-limiting range for a therapeutically effective amount of an inhibitor of the present invention is about 0.1-100 mg/kg, such as about 0.1-50 mg/kg, for example about 0.1-20 mg/kg, such as about 0.1-10 mg/kg, for instance about 0.5, about such as 0.3, about 1, about 3 mg/kg, about 5 mg/kg or about 8 mg/kg. An exemplary, non-limiting range for a therapeutically effective amount of an inhibitor of the present invention is 0.02-100 mg/kg, such as about 0.02-30 mg/kg, such as about 0.05-10 mg/kg or 0.1-3 mg/kg, for example about 0.5-2 mg/kg. Administration may e.g. be intravenous, intramuscular, intraperitoneal, or subcutaneous, and for instance administered proximal to the site of the target. Dosage regimens in the above methods of treatment and uses are adjusted to provide the optimum desired response (e.g., a therapeutic response). For example, a single bolus may be administered, several divided doses may be administered over time or the dose may be proportionally reduced or increased as indicated by the exigencies of the therapeutic situation.

In some embodiments, the efficacy of the treatment is monitored during the therapy, e.g. at predefined points in time. In some embodiments, the efficacy may be monitored by visualization of the disease area, or by other diagnostic methods described further herein, e.g. by performing one or more PET-CT scans, for example using a labeled inhibitor of the present invention, fragment or mini-antibody derived from the inhibitor of the present invention. If desired, an effective daily dose of a pharmaceutical composition may be administered as two, three, four, five, six or more sub-doses administered separately at appropriate intervals throughout the day, optionally, in unit dosage forms. In some embodiments, the human monoclonal antibodies of the present invention are administered by slow continuous infusion over a long period, such as more than 24 hours, in order to minimize any unwanted side effects. An effective dose of an inhibitor of the present invention may also be administered using a weekly, biweekly or triweekly dosing period. The dosing period may be restricted to, e.g., 8 weeks, 12 weeks or until clinical progression has been established. As non-limiting examples, treatment according to the present invention may be provided as a daily dosage of an inhibitor of the present invention in an amount of about 0.1-100 mg/kg, such as 0.2, 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of days 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of weeks 1, 2, 3, 4, 5, 6, 7, 8, 9, 10, 11, 12, 13, 14, 15, 16, 17, 18, 19 or 20 after initiation of treatment, or any combination thereof, using single or divided doses every 24, 12, 8, 6, 4, or 2 hours, or any combination thereof.

Typically, active agent is administered to the patient in the form of a pharmaceutical composition which comprises a pharmaceutically acceptable carrier. Pharmaceutically acceptable carriers that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. For use in administration to a patient, the composition will be formulated for administration to the patient. The compositions of the present invention may be administered orally, parenterally, by inhalation spray, topically, rectally, nasally, buccally, vaginally or via an implanted reservoir. The used herein includes subcutaneous, intravenous, intramuscular, intra-articular, intra-synovial, intrasternal, intrathecal, intrahepatic, intralesional and intracranial injection or infusion techniques. Sterile injectable forms of the compositions of this invention may be aqueous or an oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation. The compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include, e.g., lactose. When aqueous suspensions are required for oral use, the active ingredient is combined with emulsifying and suspending agents. If desired, certain sweetening, flavoring or coloring agents may also be added. Alternatively, the compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols. The compositions of this invention may also be administered topically, especially when the target of treatment includes areas or organs readily accessible by topical application, including diseases of the eye, the skin, or the lower intestinal tract. Suitable topical formulations are readily prepared for each of these areas or organs. For topical applications, the compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water. Topical application for the lower intestinal tract can be effected in a rectal suppository formulation (see above) or in a suitable enema formulation. Patches may also be used. The compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. For example, an antibody present in a pharmaceutical composition of this invention can be supplied at a concentration of 10 mg/mL in either 100 mg (10 mL) or 500 mg (50 mL) single-use vials. The product is formulated for IV administration in 9.0 mg/mL sodium chloride, 7.35 mg/mL sodium citrate dihydrate, 0.7 mg/mL polysorbate 80, and Sterile Water for Injection. The pH is adjusted to 6.5. An exemplary suitable dosage range for an antibody in a pharmaceutical composition of this invention may between about 1 mg/m² and 500 mg/m². However, it will be appreciated that these schedules are exemplary and that an optimal schedule and regimen can be adapted taking into account the affinity and tolerability of the particular antibody in the pharmaceutical composition that must be determined in clinical trials. A pharmaceutical composition of the invention for injection (e.g., intramuscular, i.v.) could be prepared to contain sterile buffered water (e.g. 1 ml for intramuscular), and between about 1 ng to about 100 mg, e.g. about 50 ng to about 30 mg or more preferably, about 5 mg to about 25 mg, of the inhibitor of the invention.

The invention will be further illustrated by the following figures and examples. However, these examples and figures should not be interpreted in any way as limiting the scope of the present invention.

### FIGURES

FIG. 1. Depletion of CD163+ TAM with targeted lipid-nanoparticles promotes tumor regression. (A) Schematic of αCD163 mAb conjugated lipid nanoparticles (LNP). (B) Therapeutic depletion of CD163+ TAMs in melanoma bearing mice. Mice (n=6 per group) were injected with 1×10⁶ YUMM1.7 cell s.c. on right flank. When tumors were approx. 5 mm×5 mm mice received treatment with αCD163-dxrLNP ((αCD163-dxr)) or ctrlIgG-dxrLNP (ctrlIgG-dxr) (2 mg/kg dxr) every 2^{nd} day for 2 weeks. PBS (vehicle) or empty αCD163-LNP (αCD163-ctl) was used a control. (C) At end point level of TAM and CD163+ TAM was analyzed by flow cytometry and frequency of live cells was calculated. Results are representative of 3 independent experiments. (D) Mice inoculated with 1×10⁶ YUMMER1.7 cells on the right flank were randomized into groups and treated from day 10 with 2 mg/kg dxr or control every 2^{nd} day for 2 weeks. Statistically significant differences were calculated using Mann-Whitney t-test; * p<0.05, ** p<0.01, *** p<0.001 and **** p<0.0001.

FIG. 2. CD163+ TAM depletion promotes anti-PD-1 resistant CTL responses. (A) Treatment of tumor bearing mice with αCD163-dxrLNP or vehicle in combination with aPD-1 mAb or isotype control IgG. (B) Mice were either treated every 2^{nd} day with αCD163-dxrLNP or vehicle for 10 days after which mice received aPD-1 mAb twice weekly. For each group n=6. ** p<0.01.

FIG. 3. Treatment study comparing efficacy of CD163+ TAM depletion with pan-macrophages depletion using αCSF1 blocking antibody. (A) Mice bearing palpable tumors were randomized into groups and treated with either αCD163-dxr (n=6) or PBS (n=4) i.v. every 2^{nd} day for 2 weeks or αCSF1 (n=6) or controls (CtrlIgG, n=6 or PBS, n=6) i.p. every 5 days. Statistically significant difference was calculated using two-way ANOVA followed by Tukey post hoc test; *** p<0.001 and **** p<0.0001. At end-point, total number of mTAM, iTAM and Mn (B) or CD4⁺ TIL and IFNγ⁺ CD8⁺ TIL (C) was analyzed by flow cytometry and calculated from frequency of live cells. Data is represented as mean+/−SEM of n=6.

### EXAMPLE

Material & Methods

Mouse Breeding and Initiation of Mouse Melanoma Models

To induce spontaneous melanoma formation²³, mice carrying conditional alleles BRAF^{CA/+}, PTEN^{lox4-5/lox4-5} and Tyr::CreER^{T2+/-} were shaved and exposed to 1 μL of 7.8 mg/mL 4-hydroxytamoxifen (4-HT) topically on the right flank of 5 weeks old mice. To induce syngeneic tumor, 8 weeks old male or female mice were injected subcutaneously on the right rear flank with 1×10⁶ Yale University Mouse Melanoma (YUMM1.7)²⁵ cells in 100 μl sterile PBS pH 7.4. Tumor size was measured using a digital caliper in x, y and z and tumor volume was calculated using the equation for volume of an ellipsoid (volume=0.5233xyz). All mice were housed at the animal facility at Centre d'immunologie Marseille-Luminy with water and food ad libitum and 12 h/12 h night/daylight cycle. All animal experiments were approved and carried out in accordance with the limiting principles for using animal in testing (the three R's, replacement, reduction and refinement) and approved by the French Ministry of Higher Education and Research.

Tumor Digestion, Flow Cytometry and Cell Sorting

Tumors for flow cytometry and FACS sorting were minced and digested in RPMI1640 with 1 mg/ml Collagenase II (Sigma), 50 μg/ml DNAseI (Roche) and 0.1% (w/v) BSA for 30 min at 37° C. with gentle agitation. Single-cell suspension was subsequently passed through 70 μm cell strainer and collected by centrifugation. For RBC lysis, cell suspension was incubated with 0.85% NH₄Cl for 2 min at RT, collected by centrifugation and resuspended in FACS buffer (1×PBS pH 7.4, 1 mM EDTA pH 8.0, 3% FCS and 0.1% NaN₃). For flow cytometry and FACS sorting single-cell suspensions from tumor were incubated at 4° C. for 10 min with the 2.4.G2 antibody followed by specified antibodies (See supplementary table 1 for details) for 30 min at 4° C. Prior to analysis cells were incubated with Sytox Blue (Thermo Fischer Scientific) to discriminate dead cell. For IFNγ intracellular staining, surface stained cells were incubated with Live/Dead fixable violet for 20 min in PBS to discriminate dead cells and subsequently fixed, permeabilized, and washed with BD Perm/Wash buffer (BD Biosciences) followed by incubation with IFNγ antibodies diluted in Perm/Wash buffer for 30 min at 4° C. Analysis was done on either LSR-2 or Fortessa X-20 flow cytometers equipped with 350 nm laser (BD Biosciences). Subsequent data analysis was done using FlowJo software V10.4 for Mac (Tree Star). Immunophenotyping analysis was done . . . . (Sup. Table 2 for antibodies)

Liposome Preparation

Long circulating liposomes encapsulating doxorubicin were essentially prepared³⁰ and modified for CD163 targeting as previously described²⁶. In short, liposome formulations were formed using the ethanol-injection method from a mixture of HSPC, mPEG2000-PE and Cholesterol (molar ratio of 55:40:5) (Lipoid GmBH, Ludwigshafen, Germany and Sigma Aldrich A/S, Glostrup, Denmark). Lipids were dissolved in EtOH at 65° C. for 15 min followed by hydration (to 10% EtOH) for 1 h at 65° C. in aqueous buffer suitable for further downstream applications. Liposomes were sized by extrusion 25 times through a 0.1 μm filter using the Avanti mini-extruder kit (Avanti Polar Lipids, AL, US) and dialyzed twice against 150 mM NaCl (0,9% NaCl) with second dialysis being over night at 4° C. Encapsulation of calcein (calLNP) was done by hydrating lipids in a 200 mM calcein (pH 7.4) solution with dialysis repeated five times to remove excess calcein. For remote loading of doxorubicin, lipid were hydrated in 300 mM (NH₄)₁HPO₃. Following extrusion and dialysis, (NH₄)₁HPO₃ containing liposomes were mixed with doxorubicin.HCl for 30 min at 65° C. at a doxorubicin:lipid ratio at 1:5. Lipid content, drug content and encapsulation efficiency was subsequently estimated from high-pressure size-exclusion chromatography (UV absorbance 210 nm) using a Dionex Ultimate3000 HPLC system (Thermo Scientific, Hvidovre, Denmark) equipped with a Ascentis C18 column (Sigma Aldrich A/S). Liposome sizes were estimated using dynamic light scattering and the DynaPro NanoStar system (Wyatt Technology Europe GmbH, Dernbach, Germany). Modification of liposomes for CD163 targeting was done as described earlier using the post-insertion method of αCD163 antibody clone 3E10B10^{26,31} or isotype control IgG (BioXcell).

In Vivo Treatments

When tumors reached a measurable size of approx. 5 mm×5 mm, mice received 2 mg/kg doxorubicin encapsulated in lipid nanoparticles (dxrLNP) conjugated to antiCD163 IgG (αCD163-dxrLNP) by retroorbital injection. As control, groups of mice received either 2 mg/kg doxorubicin in dxrLNP conjugated to Isotype control IgG (CtrlIgG-dxrLNP) or equivalent amount of empty LNPs conjugated to αCD163 (αCD163-LNP) or vehicle (sterile PBS pH 7.4). Mice were treated every 2 days for approximately 14 days. For in vivo experiments with calcein loaded LNPs, mice received a single injection 100 μl of a 0.67 mM lipid solution. For CD4⁺ and CD8⁺ T-cell depletion studies and anti-PD-1 mAb treatment mice received 250 μg i.p of either αCD4 (Clone GK1.5), αCD8b (clone 53-5.8), aPD-1 (clone RMP1-14) or isotype control IgG (IgG1 or IgG2a) (all BioXcell) twice a week with first injection being 1 day prior to treatment with αCD163-dxrLNP.

Immunohistochemistry and Immunofluorescence

5 mm slices of full tumor or back skin were fixed in 4% formalin and embedded in either agarose for vibratome sectioning, OCT for cryostat sectioning or paraffin for histology. For immunofluorescence of vibratome and cryostat sections 200 μm or 10 μm thick section, respectively, were cut and incubated with pAb rabbit anti-CD163-ATT056532, CD146-Alexa647 (Clone ME-9F1; BD Bioscience), CD3e-APC (Clone 145-2C11; BD Bioscience) and CD8b-FITC (53-5.8; BD Bioscience) together with anti-FITC A488 (A11096; Life Technologies) in either 0.1M Tris pH 7.2, 1% Triton X-100, 0.5% BSA for vibratome sections or in 1×PBS, 2% BSA for cryostat sections. Nuclei were visualized with Hoechst 33342 (Sigma Aldrich). Images were acquired on a Zeiss LSM780 confocal microscope using spectral unmixing and a 20× objective. For IHC section were stained with H&E and pAb rabbit anti CD163.

High-Throughput Gene Expression Analysis

Total RNA was purified from sorted population using the RNeasy Micro Kit (Qiagen) and concentration determined using the Quant-IT RiboGreen RNA assay kit (Thermo Fischer). First strand cDNA synthesis was done using a High Capacity cDNA Reverse Transcriptase Kit (Applied Biosystems) followed by preamplification of genes of interest using the Fluidigm PreAmp Master Mix (Fluidigm Corporation) using 2.5 ng of total RNA and in accordance with the manufactures instructions. Exon-spanning primers designed to amply genes of interest were calculated using Primer-Blast (see Supplementary Table 3 for details). Forward and reverse primers of genes of interest were combined to obtain gene specific assay. To increase sensitivity, genes of interest were pre-amplified by 14 cycles of PCR using pooled assays followed by exonuclease I treatment (New England Biolabs) to remove unincorporated primers. Final pre-amplified cDNA was diluted 1:5 in TE buffer. High throughput Gene expression analysis was carried out using the 96.96 dynamic arrays and Biomark HD system from Fluidigm (Fluidigm Europe B.V.) in accordance with manufactures instructions and standard settings. Obtained data was analyzed using the Real-Time PCR Analysis Software (Fluidigm Europe B.V.) and resulting CT values were normalized to Cph to obtain dCT values. Heatmaps, Z-scores and hierarchical clustering using the One minus pearson correlation were generated using Morpheus (https://software.broadinstitute.org/morpheus/). PCA plots were generated using Qlucore Omics (Qlucore AB, Lund, Sweden).

Statistical Analysis

For treatment studies statistical analysis was done using two-way ANOVA followed by Tukey post hoc test. For comparison between groups statistical testing was done using non-parametric test such as Mann-Whitney or Kruskal-Wallis where appropriate. Data was consider statistical significant when p<0.05. p values are indicated as * p<0.05, ** p<0.01, *** p<0.001 and **** p<0.0001. All statistical analysis was done in Graphpad Prism 7 for Mac.

Results

CD163-Expressing Macrophages Infiltrate Spontaneous Braf^{V600E}-Driven Melanomas

Activating mutations in BRAF are the most prevalent in human melanoma, often accompanied by loss of tumor suppressor genes such as PTEN and CDKN2A. The increased availability of genetically engineered mouse (GEM) models based on the appropriate oncogenic driver mutations has greatly improved the relevance of mouse tumor models to human disease. The Tyr::CreER; Braf^{CA}; Pten^{f/f} mouse model of metastatic melanoma utilizes the melanocyte-restricted tyrosinase (Tyr) promoter to drive expression of a tamoxifen-inducible Cre-recombinase (CreER^{T2}), which in-turn, triggers expression of constitutively active Braf^{V600E} (Braf^{CA}) and deletion of a floxed Pten allele (Pten^{f/f})²³. In these mice, sub-cutaneous (s.c.) administration of 4-hydroxytamoxifen (4-HT) initially leads to small pigmented lesions from around day 20, which progress to amelanotic tumors at around day 40 that subsequently exhibit exponential growth (data not shown). In untreated mice, CD163⁺ macrophages are evenly dispersed throughout the dermis and adipose tissue (data not shown). However, after 4-HT treatment CD163⁺ macrophages accumulate at the border of pigmented, pre-melanotic lesions in the dermis (data not shown). When pigmented lesions transform into fast growing amelanotic tumors, CD163⁺ macrophages accumulate at the invasive front, whereas only few CD163⁺ macrophages are present within the tumor (data not shown). To further examine the tumor-infiltrating myeloid cell (TIM) compartment in Braf^{V600E} tumors, we performed flow cytometry on single-cell suspensions of tumor tissue. After gating out CD45-negative tumor cells, lymphocytes and granulocytes (CD45.2⁺, CD19⁻, CD5⁻, NK1.1⁻, SiglecF⁻, Ly6G⁻ and CD11b⁺), we found two major populations of monocytes/macrophages based on F4/80 and CD169 expression. The F4/80⁻ CD169⁻ population consisted mainly of Ly6C⁺ monocytes (MN) and Ly6C⁺ MHCII⁺ immature macrophages (intTAM), as previously described in other models²⁴. The larger F4/80⁺ CD169⁺ population, was negative for Ly6C and showed heterogeneous expression of CD163 and MHCII, suggesting a mature tumor-associated macrophage (TAM) phenotype (data not shown). Overall, TAM were by far the most abundant cell type, making up 60% of the CD11b⁺ fraction (data not shown). On average, 20% of all tumor-infiltrating leukocytes were TAM, which is 100 times more than the number of tumor-infiltrating CD8⁺ T cells (data not shown). CD163⁺ macrophages made up only <25% of all TAM and could be separated into a more abundant MHCII-population and a minor population of MHCII⁺ cells.

Characterization of CD163-Expressing TAM in Orthotropic Braf^{V600E}-driven melanomas.

Using cell lines derived from the Braf^{V600E}-driven mouse model, that mimic the amelanotic stage of tumor growth²⁵, we sought to determine if implanted orthotropic tumors with comparable driver-mutations would give rise to similar TAM populations found in the spontaneous amelanotic tumors. The YUMM1.7 cell line (Yale University Mouse Melanoma), is derived from a spontaneous melanoma driven by Braf^{V600E} activation and inactivation Pten and Cdkn2a. YUMM1.7 cells give rise to tumors with similar growth characteristics as spontaneous Braf^{V600E} tumors (data not shown). Flow cytometry analysis of YUMM1.7 tumors revealed distinct tumor-associated monocyte/macrophage populations based on F4/80 and CD169 expression, similarly to spontaneous Braf^{V600E} tumors. However, whereas F4/80-CD169⁻ and F4/80⁺ CD169⁺ populations seemed identical in the two models, there was also a pronounced F4/80⁻ CD169⁺ population present in orthotropic tumors (data not shown). This population consisted mainly of the intTAM, as described in the spontaneous model (data not shown), in addition to a few Ly6C⁺ MN (data not shown). Accordingly, a higher proportion of MN (F4/80⁻ CD169⁻) and intTAM are present in early tumors, whereas the proportion of mature TAM (F4/80⁺ CD169⁺) steadily increases, to become the most abundant in late-stage tumors (data not shown). Interestingly, the orthotropic tumor was infiltrated by both a CD163^{hi} and CD163^{lo} TAM, that are mostly MHCII-negative (data not shown). Immunohistological (IHC) analysis, suggested CD163^{hi/lo} TAM differ in their spatial distribution; CD163^{hi} cells were mainly localized at the tumor margin, whereas CD163^{lo} cells were located inside the tumor tissue (data not shown). In addition, the proportion of MHCII⁻ CD163^{lo} TAM increases with tumor progression, whereas the proportion of MHCII⁻ CD163^{hi} cells remained constant (data not shown).

To further characterize the different monocyte/macrophage populations in this melanoma model, we isolated MN, intTAM and four mature TAM populations, by flow cytometry and performed high-density quantitative PCR (qPCR) analysis of gene expression using the Fluidigm Biomark system (data not shown). We analyzed both CD163^{hi/lo} TAM (MHCII⁻ CD163^{lo}; MHCII⁻ CD163^{hi}) and CD163-negative TAM (MHCII⁻ CD163⁻; MHCII⁺ CD163⁻), as well as MN and intTAM populations (data not shown). Gene expression data was analyzed by hierarchical clustering and principal component analysis, which revealed a cluster of primarily M2-macrophage associated genes upregulated in CD163-expressing TAM subsets (cluster III), including; Il4ra, Mrc1, Stab1, Slco2b1 (data not shown). Interestingly, especially in the CD163^{hi} subset, there was also an upregulation of genes that are known to be associated with effects on CD8⁺ T cell function, including Il10, Ido1 and Lgals1 (data not shown).

We also observed distinctive clusters of inflammatory genes upregulated in tumor-associated monocytes (MN) (cluster I; inc. Cxcl10, Il1b, Irf5, Ccr2, Il18) and intTAM (cluster II; inc. Cxcl9, Ciita, Irf7), these genes were downregulated in mature TAM, coincident with acquisition on an M2-like phenotype (cluster III). Many of the genes represented in clusters I and II are IFN-responsive and reflect a M1-like phenotype, normally associated with immune-stimulatory activity. This, in combination with the principal component and network analysis (data not shown), suggest that recruited MN are progressively polarized towards a M2-like TAM phenotype. This analysis also showed high expression of Nr4a1 in tumor-infiltrating MN. The nuclear receptor Nr4a1 (Nur77) is generally regarded as a marker for so-called patrolling or non-classical monocytes, as high expression is normally only detected on Ly6C⁻ monocytes in the circulation (data not shown). To further investigate Nr4a1 expression in tumor-associated MN, we analyzed YUMM1.7 tumors from Tg(Nr4a1-GFP) mice; flow cytometry analysis showed comparable levels of GFP expression in Ly6C⁺ tumor-associated MN and patrolling (Ly6C^{lo}) monocytes in the blood (data not shown), which was progressively reduced in intTAM and mature TAM, respectively (data not shown). Suggesting that tumor-associated MN may be derived from a population of non-classical monocytes in the circulation, which downregulate Nr4a1 expression as they differentiate into mature TAM.

Specific Depletion of CD163-Expressing TAM with Targeted Lipid-Nanoparticles Promotes Tumor Regression.

To deplete CD163⁺ TAM we generated knock-in mice expressing iCre recombinase from a Cd163-IRES-iCre transcript (CD163-iCre) and crossed them to Csflr-LSL-DTR mice that after cre-mediated deletion of LSL, have DTR expression under the control of the Csflr promotor. A single injection of 4 ng/kg diphteria toxin (DT) specifically depleted close to 50% of CD163+ TAMs after 24 hrs (data not shown). No effect, was observed on the remaining myeloid compartment except for a small increase in recruited monocytes (data not shown). Next, we attempted to achieve sustained depletion of CD163+ TAMs by repeated DT injections. Despite a significantly reduced tumor growth in CD163-iCre^{+/-}, Csf1r-LSL-DTR^{+/-} mice compared to Csflr-LSL-DTR wildtype mice (data not shown), sustained depletion of CD163⁺ macrophages caused severe side-effect and proved non-suitable for continuous use (data not shown).

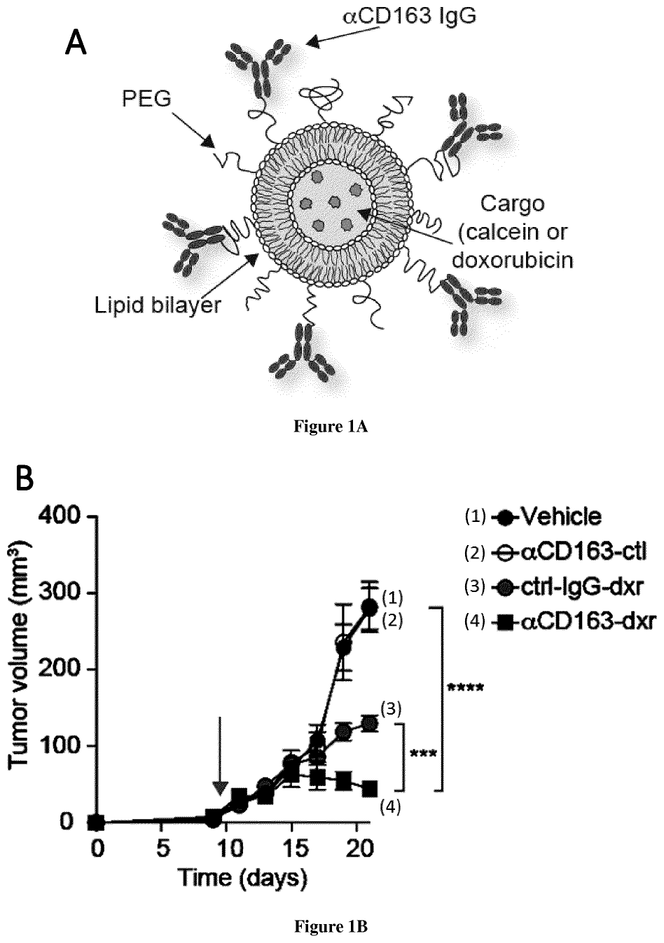

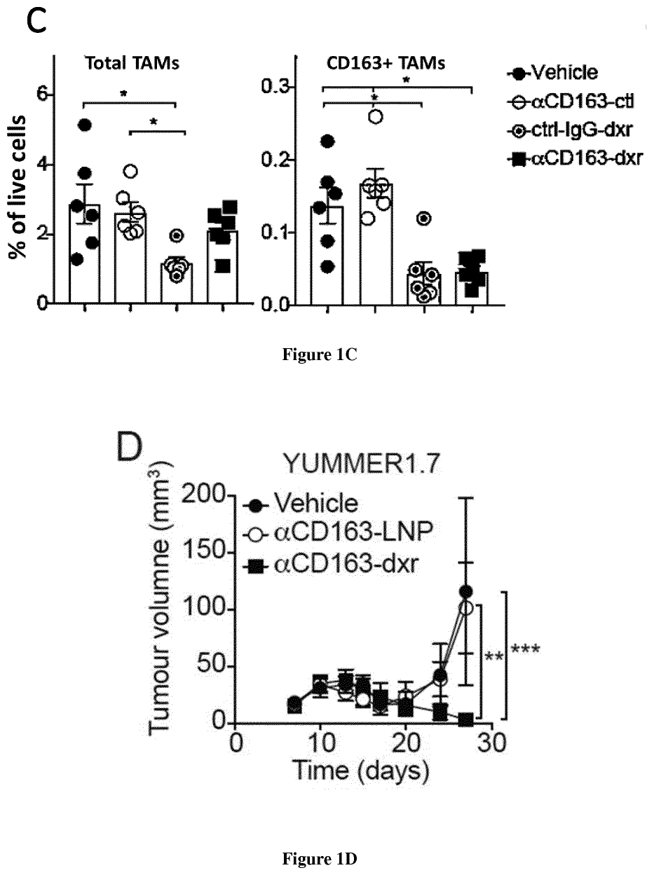

We previously developed a method for specific targeting of CD163-expressing cells using anti-CD163 mAb-conjugated lipid-nanoparticles (LNP)²⁶ (FIG. 1A). These LNPs contain 5% polyethylene glycol (PEG; 2000 mw) that minimizes non-specific phagocytic uptake and increases the specificity of targeting to CD163⁺ cells. The anti-CD163 mAb is incorporated into the LNP via a polyethylene (3400 mw) lipid anchor that is covalently attached to Lysine side-chains of the antibody (αCD163-LNP). To illustrate the specificity of αCD163-LNPs, we used recombinant CHO K1 cells expressing mouse CD163 (data not shown) and mouse thioglycolate-elicited macrophages (data not shown); αCD163-LNPs were loaded with self-quenching concentrations of calcein (αCD163-cal-LNP; αCD163-cal) to monitor cellular uptake, controls included calcien loaded LNPs alone (cal-LNP) and LNPs conjugated with an isotype-control Ab (ctrl-IgG-cal-LNP; IgG-cal). Due to the encapsulation of self-quenching concentrations of calcein, fluorescence is only observed when calcein-loaded LNPs are taken-up by endocytosis and the LNP is degraded in the endolysosomal compartment of target cells. No increase in calcein fluorescence was observed when non-targeted LNPs (cal-LNP or ctrl-IgG-cal-LNP) were incubated with CD163-expressing CHO K1 cells (data not shown) and only a minor increase was observed with ctrl-IgG-cal-LNP in primary mouse macrophages (data not shown). These assays showed that high calcein fluorescence was specific for CD163-expressing cells upon incubation with αCD163-cal-LNP. To evaluate the targeting of αCD163-LNPs to CD163-expressing TAM in our melanoma model, αCD163-LNPs and non-targeted control LNPs were injected intra-venously (i.v.) in tumor-bearing mice followed by in vivo fluorescence imaging; 4 hours after injection, fluorescence could be detected in the tumor area with both αCD163-targeted LNPs and non-targeted LNPs (data not shown). However, analysis of tumor tissue by flow cytometry showed increased calcien uptake in CD163′ TAM after administration of αCD163-LNP, compared to non-targeted LNPs (data not shown). We next generated cytotoxic LNPs to test the ability of αCD163-targeted LNPs to specifically deplete CD163⁺ macrophages in vivo. We loaded LNPs with the DNA damaging agent doxorubicin (dxr) and injected randomized groups of mice i.v. with a single dose of either vehicle, empty αCD163-LNP (αCD163-ctrl) or dxr-loaded targeted and non-targeted LNPs (αCD163-dxr or IgG-dxr, respectively), 24 hours later we measured the effects on CD163′ red pulp macrophages (RPM) in the spleen by flow cytometry; a single injection of αCD163-dxr specifically reduced the number of CD163′ RPM by approximately 50%, compared to controls (data not shown). Next, we tested the effects of CD163′ TAM depletion on tumor growth in melanoma-bearing mice. To achieve an efficient and sustained depletion of CD163′ TAM, mice with palpable tumors were randomized and treated every 2^{nd} day for 2 weeks with αCD163-dxr or appropriate controls. Although treatment with non-targeted cytotoxic LNPs (IgG-dxr) was able to slow tumor growth, mice treated with αCD163-dxr showed almost complete tumor regression after 2 weeks (FIGS. 1B and 1D). Interestingly, subsequent flow cytometry analysis of tumors showed a reduction of total TAM numbers in mice treated with IgG-dxr (FIG. 1C), suggesting indiscriminate pan-targeting of TAM subsets. However, CD163-targeted LNPs only depleted the minor fraction of CD163⁺ TAM, having little impact on total TAM numbers (FIG. 1C). Given the profound effects of CD163-targeted LNPs on tumor regression, even compared to non-targeted LNPs, this implied that pan-targeting of TAM subsets may in fact abrogate the therapeutic effects conferred by the depletion of CD163⁺ TAM. Suggesting that other TAM subsets contribute to tumor regression upon CD163⁺ TAM depletion.

Targeted Depletion of CD163⁺ TAM re-educates tumor-infiltrating myeloid cells.

To further analyze the consequence of CD163⁺ TAM depletion on the tumor-immune microenvironment (TME), we performed a high-content immunophenotyping by flow cytometry on tumors after treatment with αCD163-dxr compared to empty CD163-targeted LNP (αCD163-ctrl) and vehicle treated mice. The depletion of CD163⁺ TAM was associated with a highly significant overall expansion of the tumor-infiltrating leukocyte compartment, increasing from 5 to 30% of all cells (data not shown). Analysis of different immune cell types revealed this was mainly due increased numbers of tumor-infiltrating T cells (TIL), both CD4⁺ and CD8⁺, and especially Ly6C⁺ monocytes (data not shown). To further characterize the tumor-infiltrating myeloid (TIM) cell compartment after CD163⁺ TAM depletion, we performed flow cytometry analysis using our previously established gating strategy (data not shown). In αCD163-dxr-treated mice, there was a dramatic increase in the recruitment of tumor-associated MN and intTAM, collectively making up more than 300 million cells per gram of tissue (data not shown), and consequently a drastic reduction in the proportion of mature TAM (data not shown). Interestingly, intTAMs that infiltrated tumors after CD163⁺ TAM depletion, showed a significant increase in CD11c expression (data not shown) and displayed a distinct gene expression profile, as compared to intTAM from control tumors (data not shown), including increased expression of Ciita and Cxcl9 (data not shown), which indicated an immune-stimulatory phenotype typical of activated monocyte-derived dendritic cells (moDC). Importantly the intTAMs from αCD163-dxrLNP treated mice showed a significantly decreased expression of genes normally associated with patrolling or non-classical monocytes such as Nr4a1 and Cx3cr1 whereas Cxcr4 expression was unchanged and Fcgr2b that is associated with classical Ly6C^{hi} monocytes was increased (data not shown). Moreover, intTAMs from αCD163-dxrLNP treated mice also showed increased expression of Pdl2 and CD209d as well as the T-cell chemokines Cxcl9 and Ccl17 (data not shown). The decreased expression of genes associated with non-classical monocytes in intTAMs from CD163⁺ TAM depleted mice points towards a re-education of tumor-infiltrating myeloid cells by a recruitment of a classical inflammatory monocyte subset.

Mobilization of inflammatory monocytes (Ly6C⁺, Nr4a1⁻, Cx3crl^{lo}), is highly dependent on expression of the chemokine receptor CCR2 and severely impaired in CCR2-deficident mice (Ccr2^{-/-}). In contrast, the distribution of patrolling monocytes (Ly6C^{lo}, Nr4a1⁺, Cx3crl^{hi}) is only slightly affected in Ccr2^{-/-} mice²⁷. To assess the contribution of CCR2-dependent monocyte recruitment to the accumulation of Ly6C⁺ MN and intTAM after CD163⁺ TAM depletion, we generated cohorts of wildtype (WT) and Ccr2^{-/-} mice bearing melanomas and treated with αCD163-dxr or vehicle, as described above. Interestingly, tumor progression in vehicle treated mice was unaffected by CCR2-deficiency (data not shown). Furthermore, accumulation of tumor-associated MN and intTAM was only marginally reduced whereas mature TAM was unaffected in Ccr2^{-/-} mice receiving vehicle treatment (data not shown). However, inhibition of tumor growth by αCD163-dxr treatment was significantly abrogated in Ccr2^{-/-} mice (data not shown), which was accompanied by a complete reversal of intTAM recruitment provoked by CD163⁺ TAM depletion (data not shown). These data showed that depletion of CD163⁺ TAM in melanoma bearing mice results in CCR2-dependent recruitment of fresh Ly6C⁺ monocytes and accumulation of immune-stimulatory macrophages with a M1-like phenotype, that significantly contribute to inhibition of tumor progression.

CD163⁺ TAM Depletion Promotes Anti-PD-1 Resistant CTL Responses

Immunophenotyping analysis of melanomas after depletion of CD163⁺ TAM showed a significant increase in tumor-infiltrating T cells (TIL) (data not shown). To further analyze the effects of CD163⁺ TAM depletion on TIL recruitment and activation we performed additional flow cytometry analysis. Gating on TIL (CD45.2⁺, CD19⁻, NK1.1⁻, SiglecF⁻, Ly6G⁻ and CD11b⁻, CD3e⁺, CD5⁺,) we confirmed a profound increase in both CD4⁺ and CD8⁺ TIL in melanoma-bearing mice treated αCD163-dxr, compared with vehicle or empty αCD163-LNP (αCD163-ctrl) (data not shown). In control treated mice, CD8⁺ TIL displayed a heterogeneous expression of IFNγ and PD-1 (data not shown), however, in mice treated with αCD163-dxr, the majority of CD8⁺ TIL expressed high levels of IFNγ and no PD-1 (data not shown). We confirmed increased infiltration of CD8⁺ TIL in melanomas by confocal microscopy in tumor sections (data not shown), which correlated with the depletion of CD163⁺ TAM (data not shown). In accordance with increased infiltration of activated CTL, the tissue-wide expression of IFNγ (Ifng) was dramatically increased in tumors upon depletion of CD163⁺ TAM (data not shown), this was accompanied with increased expression of other inflammatory cytokines, including TNFα, IL-1β (Il1b) and IL-18 (Il18) (data not shown), as well as the increased expression of the memory T-cell attracting chemokine CXCL9 (Cxcl9) (data not shown).

The tissue-wide increase in Cxcl9 expression upon depletion of CD163⁺ TAM, correlated with a parallel increase in Cxcl9 expression in freshly recruited intTAM (data not shown). CXCL9 is a potent chemoattractant for memory CD8⁺ T cells and induction of Cxcl9 expression in antigen-presenting cells (APCs) by T cell-derived IFNγ, has been shown to be critical for the propagation of CTL responses. This led us to investigate if the recruitment of CCR2-dependent intTAM, induced by CD163⁺ TAM depletion (data not shown), was connected to the observed changes in the TIL compartment. We analysed TIL in melanomas from WT and Ccr2^{-/-} mice treated with αCD163-dxr or vehicle alone by flow cytometry; CD163⁺ TAM depletion in both WT and Ccr2^{-/-} mice led to an increase in CD4⁺ and CD8⁺ TIL (data not shown), however, CD4⁺ TIL and particularly the number of IFNγ-producing CD8⁺ TIL, were significantly reduced in Ccr2^{-/-} mice compared to WT mice (data not shown). These data suggested that the CCR2-dependent recruitment of intTAM, after depletion of CD163⁺ TAM, contributed to the recruitment and activation of CD4⁺ and CD8⁺ TIL, respectively.

To establish the contributions of CD4⁺ and CD8⁺ TIL to tumor regression upon depletion of CD163+ TAM, we administrated αCD4 and αCD8b mAbs during treatment with αCD163-dxr to deplete CD4⁺ and CD8⁺ TIL. Both CD4⁺ and CD8⁺ T cell depletion completely reversed the control of tumor growth by αCD163-dxr-treatment in melanoma-bearing mice (data not shown). Clearly demonstrating that the inhibition of tumor progression upon depletion of CD163⁺ TAM was driven by activation of the TILs. Interestingly, depletion of CD4⁺ TIL alone, markedly reduced the number of infiltrating intTAM and IFNγ-producing CD8⁺ TIL after depletion of CD163′ TAM (data not shown). In addition, gene expression analysis showed that depletion of both CD4⁺ and CD8⁺ T cells reduced global Ifng expression whereas global expression of Cxcl9 and Il1b was only reduced in CD4⁺ T-cell depleted mice (data not shown).

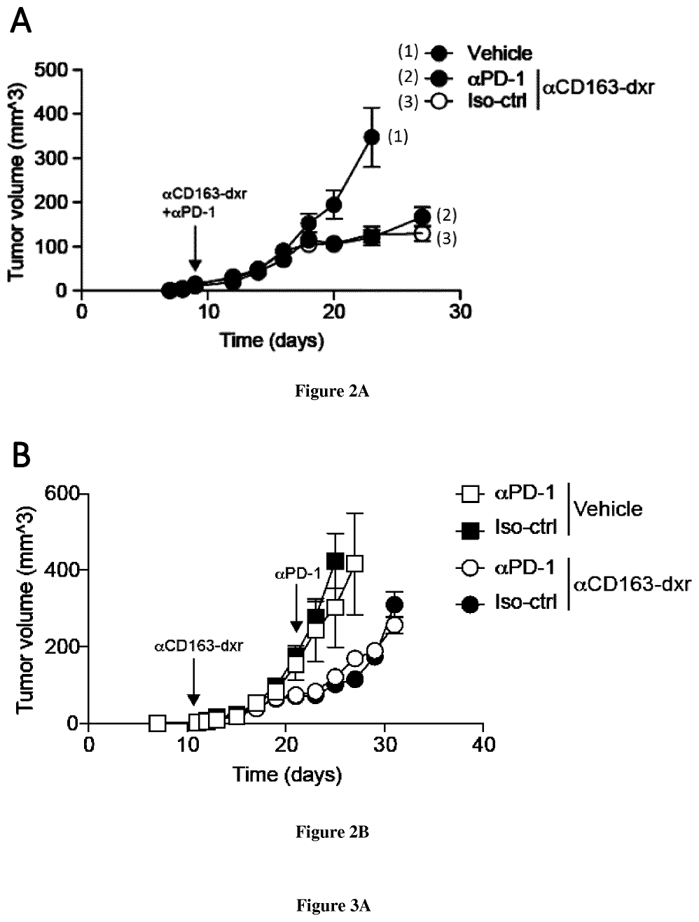

Given the expression of PD-L1 on YUMM1.7 cells²⁸ and increased expression of PD-L2 on intTAMs after αCD163-dxr treatment (data not shown) we sought to investigate if the observed treatment effect was affected by PD-1 checkpoint inhibition. We administered aPD-1 mAb or control mAb to tumor-bearing mice, either alone, in combination with αCD163-dxr for 10 days (FIG. 2A) or following αCD163-dxr treatment (FIG. 2B). As shown in previous studies, αPD-1 mAb treatment alone, had no effect on the growth of YUMM1.7 tumors (FIG. 2A). Furthermore, while αCD163-dxr efficiently controlled tumor growth with or without concomitant treatment with aPD-1 (FIG. 2A), when αCD163-dxr was substituted for αPD-1 alone, tumors quickly relapsed (FIG. 2B).

Treatment Study Comparing Efficacy of CD163+ TAM depletion with pan-macrophages depletion using αCSF1 blocking antibody

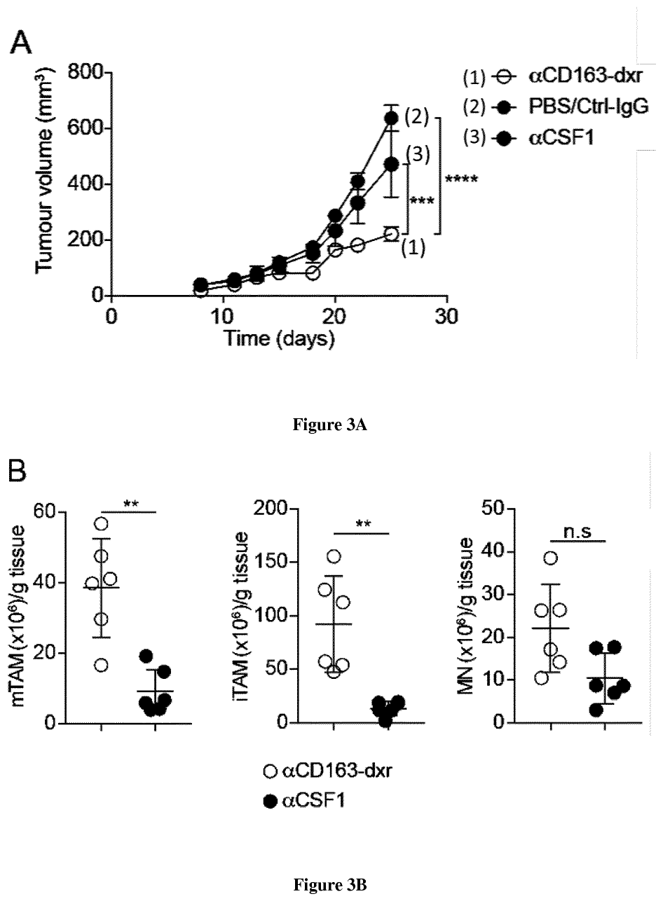

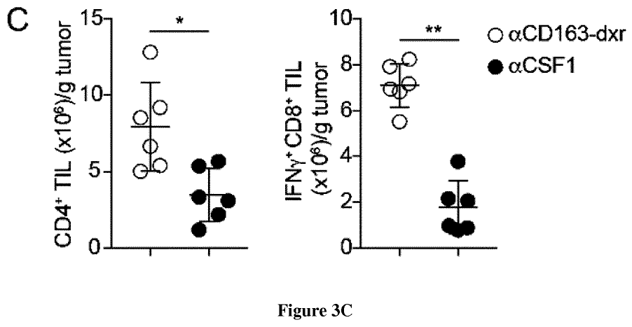

To compare the efficacy of CD163+ TAM depletion with pan-macrophages using αCSF1 blocking antibody, we treated mice bearing palpable tumors with either αCD163-dxr every 2^{nd} day for 2 weeks or αCSF1 (n=6) i.p. every 5 days. The pan-depletion of TAM subsets using anti-CSF1 resulted in a less pronounced inhibition of tumor growth compared with specific targeting of CD163+ TAMs (FIG. 3A). Interestingly, anti-CSF1 treatment was associated with a strong reduction in all TAM subsets, including bone marrow-derived monocytes (MNs) (FIG. 3B). Moreover, the reduction of iTAM after anti-CSF1 treatment was associated with a reduced number of tumor-infiltrating CD4+ and IFNγ-producing CD8+ T cells as compared with specific depletion of CD163+ TAMs (FIGS. 3B and 3C).

### Discussion

There is now ample experimental and clinical evidence highlighting the many important disease-promoting functions of tumor-associated macrophages (TAM), emphasizing their key role in progression of cancer. Consequently, the interest in developing novel therapeutic strategies to target TAM is increasing. To date, most strategies have focused on CSF-1/CSF1R-signaling that regulates macrophage differentiation and survival or the CCL2/CCR2 axis, which regulates monocyte mobilization and recruitment. However, these strategies have shown limited effects in experimental models and clinical trials. Recent research has exposed an extensive heterogeneity among TAM subsets in human cancers, which may have important implications for clinical progression. Indeed, several clinical studies have shown that certain TAM subsets can be associated with good prognosis in patients. Hence more data is a needed to further understand the function of specific TAM subsets to aid the future development of more targeted therapies.

Despite their intrinsic immune-stimulatory potential, one of the major tumor-promoting functions of TAM is thought to be immune-suppression. It has been suggested that the tumor-microenvironment (TME) polarizes macrophages towards an alternative activation state, associated with suppression of tumor-infiltrating T cell (TIL) function, rather than activation. Immune-checkpoint inhibitors (ICI), which trigger activation of TILs, have made an unprecedented impact on the treatment of certain cancers, particularly malignant melanoma. However, the majority of patients still do not respond to existing ICI therapies which often correlates with low levels of TILs in primary tumors.

We chose to investigate the role of TAM in a clinically relevant mouse models of melanoma which is resistant to the current leading ICI therapy, anti-PD-1. We first characterized the tumor-infiltrating myeloid (TIM) compartment in both autochthonous and orthotopic melanomas; TAM constituted up to 60% of CD11b⁺ leukocytes in tumors and on average 20% of all tumor-infiltrating leukocytes were TAM, which was 100 times more than the number of CD8⁺ tumor-infiltrating T cells (TIL). Clinical data has strongly linked expression of CD163 by TAM and poor prognosis in a range of cancers, including melanoma. However, the functional relevance of these cells in tumor progression is still unclear. CD163⁺ TAM represented only a minor fraction of all TAM in mouse melanomas (<25%). Gene expression analysis of CD163′ TAM revealed the upregulation of a cluster of genes associated with M2-like macrophages (including Il4ra, Mrc1, Stab1, Slco2b1). Interestingly, there was also a specific upregulation of genes that are known to inhibit T cell activation, including 1110, Ido1 and Lgals1. This was in contrast to distinctive clusters of inflammatory genes upregulated in tumor-infiltrating monocytes (MN) (such as Cxcl10, Il1b, Irf5, Ccr2, Il18) and immature TAM (intTAM; Cxcl9, Ciita, Irf7), these genes were downregulated in CD163⁺ TAM, coincident with acquisition on an M2-like phenotype. Many of the genes upregulated in MN and intTAM, were IFN-responsive and reflect a M1-like phenotype, normally associated with immune-stimulatory activity and anti-tumor functions. This suggests recruited MN are progressively polarized towards a M2-like TAM phenotype.

To assess the specific contribution of CD163⁺ TAM to tumor progression, we developed CD163-targeted cytotoxic lipid-nanoparticles (LNPs). CD163-targeted LNPs only depleted the minor fraction of CD163⁺ TAM, having little impact on total TAM numbers. However, the selective depletion of CD163⁺ TAM profoundly reduced tumor growth. Interestingly, we observed that non-targeted cytotoxic LNPs, that significantly reduced total TAM numbers, were not as effective as CD163-targeted LNPs in reducing tumor growth. This implied that pan-targeting of TAM subsets may in fact abrogate the therapeutic effects conferred by the specific depletion of CD163⁺ TAM. This suggests that other TAM subsets could contribute to tumor regression upon CD163⁺ TAM depletion. To explore this hypothesis, we assessed the impact of CD163⁺ TAM depletion on the TIM compartment in melanomas. We observed that intTAM, which infiltrated tumors after CD163⁺ TAM depletion, were CD11c^{hi} and had increased expression of Ciita, Cxcl9 and CD209d, indicating an immune-stimulatory phenotype typical of monocyte-derived dendritic cells (moDC)²⁹. Recruitment of CD11c^{hi} intTAM was blocked in CCR2-deficient mice, which abrogated the reduction in tumor growth induced by CD163⁺ TAM depletion. Thus, the mobilization of CCR2-dependent inflammatory monocytes significantly contributed to tumor regression. CD163⁺ TAM depletion also increased the numbers of CD4⁺ and CD8⁺ TIL in melanomas, both of which were required for controlling tumor growth. Interestingly, both CD4⁺ and CD8⁺ TIL recruitment was CCR2-dependent, indicating a requirement for recruitment of inflammatory monocytes and likely the accumulation of CD11c^{hi} intTAM. This was perhaps mediated by the increased expression of CXCL9 in these cells, a critical chemokine for recruitment of memory T cells, coupled with enhanced antigen-presenting cell (APC) activity associated with increased MHC II expression via Ciita. As expected, blockade of CD4⁺ TIL accumulation, after depletion of CD163⁺ TAM, markedly reduced the number IFNγ-producing CD8⁺ TIL—in keeping with the role of CD4⁺ T cell help for CD8⁺ TIL activation. However, depletion of CD4⁺ TIL, but not CD8⁺ TIL, also significantly reduced the number of infiltrating CD11c^{hi} intTAM. Suggesting that CD4⁺ TIL specifically contribute to inflammatory monocyte mobilization which may in-turn promote recruitment and activation of CD8⁺ TIL.

In summary, our studies demonstrate a profound immune-suppressive function for CD163⁺ TAM in melanomas. Furthermore, the specific depletion of CD163⁺ TAM allows the reprogramming of tumor-infiltrating monocytes towards immune-stimulatory functions and tumor regression. These data not only present a new therapeutic strategy based on the specific targeting of TAM subsets, but also explain the lack of efficacy with therapeutic approaches that indiscriminately targeted monocyte-derived macrophages. Thus, the requirement for inflammatory monocytes to propagate TIL recruitment and activation may limit the utility of pan-monocyte/macrophage targeted therapies.

### REFERENCES

Throughout this application, various references describe the state of the art to which this invention pertains. The disclosures of these references are hereby incorporated by reference into the present disclosure.
1. Noy, R. & Pollard, J. W. Tumor-Associated Macrophages: From Mechanisms to Therapy. Immunity 41, 49-61 (2014).
2. Jiang, Y., Li, Y. & Zhu, B. T-cell exhaustion in the tumor microenvironment. Cell Death Dis 6, e1792-e1792 (2015).
3. Rodriguez, P. C. et al. Arginase I Production in the Tumor Microenvironment by Mature Myeloid Cells Inhibits T-Cell Receptor Expression and Antigen-Specific T-Cell Responses. Cancer Res 64, 5839-5849 (2004).
4. Munn, D. H. & Mellor, A. L. Indoleamine 2,3-dioxygenase and tumor-induced tolerance. J. Clin. Invest. 117, 1147-1154 (2007).
5. Li Yang, Y. Z. Tumor-associated macrophages, potential targets for cancer treatment. Biomarker Research 5, 1423 (2017).
6. Wynn, T. A., Chawla, A. & Pollard, J. W. Macrophage biology in development, homeostasis and disease. Nature 496, 445-455 (2013).
7. Cassier, P. A. et al. CSF1R inhibition with emactuzumab in locally advanced diffuse-type tenosynovial giant cell tumours of the soft tissue: a dose-escalation and dose-expansion phase 1 study. The Lancet Oncology 16, 949-956 (2015).
8. Ries, C. H. et al. Targeting Tumor-Associated Macrophages with Anti-CSF-1R Antibody Reveals a Strategy for Cancer Therapy. Cancer Cell 25, 846-859 (2014).
9. de Vos van Steenwijk, P. J. et al. Tumor-infiltrating CD14-positive myeloid cells and CD8-positive T-cells prolong survival in patients with cervical carcinoma. Int. J. Cancer 133, 2884-2894 (2013).
10. Ino, Y. et al. Immune cell infiltration as an indicator of the immune microenvironment of pancreatic cancer. Br. J. Cancer 108, 914-923 (2013).
11. Mantovani, A. & Allavena, P. The interaction of anticancer therapies with tumor-associated macrophages. J. Exp. Med. 212, 435-445 (2015).
12. Chevrier, S. et al. An Immune Atlas of Clear Cell Renal Cell Carcinoma. Cell 169, 736-749.e18 (2017).
13. Lavin, Y. et al. Innate Immune Landscape in Early Lung Adenocarcinoma by Paired Single-Cell Analyses. Cell 169, 750-765.e17 (2017).
14. Kristiansen, M. et al. Identification of the haemoglobin scavenger receptor.

Nature 409, 198-201 (2001).
15. Etzerodt, A. & Moestrup, S. K. CD163 and inflammation: biological, diagnostic, and therapeutic aspects. Antioxid. Redox Signal. 18, 2352-2363 (2013).
16. Etzerodt, A., Maniecki, M. B., Møller, K., Møller, H. J. & Moestrup, S. K.

Tumor necrosis factor α-converting enzyme (TACE/ADAM17) mediates ectodomain shedding of the scavenger receptor CD163. J Leukoc Biol 88, 1201-1205 (2010).
17. Ugurel, S. et al. Survival of patients with advanced metastatic melanoma: the impact of novel therapies-update 2017. Eur J Cancer 83, 247-257 (2017).
18. Robert, C. et al. Pembrolizumab versus Ipilimumab in Advanced Melanoma. http://dx.doi.org/10.1056/NEJMoa1503093 372, 2521-2532 (2015).
19. Pardoll, D. M. The blockade of immune checkpoints in cancer immunotherapy. Nat. Rev. Cancer 12, 252-264 (2012).
20. Tumeh, P. C. et al. PD-1 blockade induces responses by inhibiting adaptive immune resistance. Nature 515, 568-571 (2014).
21. Larkin, J. et al. Combined Nivolumab and Ipilimumab or Monotherapy in Untreated Melanoma. N. Engl. J. Med. 373, 23-34 (2015).
22. Michot, J. M. et al. Immune-related adverse events with immune checkpoint blockade: a comprehensive review. Eur J Cancer 54, 139-148 (2016).
23. Dankort, D. et al. BrafV600E cooperates with Pten loss to induce metastatic melanoma. Nature genetics 41, 544-552 (2009).
24. Movahedi, K. et al. Different Tumor Microenvironments Contain Functionally Distinct Subsets of Macrophages Derived from Ly6C(high) Monocytes. Cancer Res 70, 5728-5739 (2010).
25. Meeth, K., Wang, J. X., Micevic, G., Damsky, W. & Bosenberg, M. W. The YUMM lines: a series of congenic mouse melanoma cell lines with defined genetic alterations. Pigment Cell Melanoma Res. 29, 590-597 (2016).
26. Etzerodt, A. et al. Efficient intracellular drug-targeting of macrophages using stealth liposomes directed to the hemoglobin scavenger receptor CD163. J Control Release 160, 72-80 (2012).
27. Tsou, C.-L. et al. Critical roles for CCR2 and MCP-3 in monocyte mobilization from bone marrow and recruitment to inflammatory sites. J. Clin. Invest. 117, 902-909 (2007).
28. Natale, C. A. et al. Activation of G protein-coupled estrogen receptor signaling inhibits melanoma and improves response to immune checkpoint blockade. eLife 7, 116 (2018).
29. Menezes, S. et al. The Heterogeneity of Ly6Chi Monocytes Controls Their Differentiation into iNOS+ Macrophages or Monocyte-Derived Dendritic Cells. Immunity 45, 1205-1218 (2016).
30. Fritze, A., Hens, F., Kimpfler, A., Schubert, R. & Peschka-Siiss, R. Remote loading of doxorubicin into liposomes driven by a transmembrane phosphate gradient. Biochim Biophys Acta 1758, 1633-1640 (2006).
31. Torchilin, V. P. et al. p-Nitrophenylcarbonyl-PEG-PE-liposomes: fast and simple attachment of specific ligands, including monoclonal antibodies, to distal ends of PEG chains via p-nitrophenylcarbonyl groups. Biochim Biophys Acta 1511, 397-411 (2001).
32. Etzerodt, A. et al. Plasma Clearance of Hemoglobin and Haptoglobin in Mice and Effect of CD163 Gene Targeting Disruption. Antioxid Redox Signal 18, 2254-2263 (2013).

1. A method of increasing the amount of tumor infiltrating CD8+ T cells in a patient suffering from cancer comprising administering to the patient a therapeutically effective amount of an antibody having binding affinity for CD163.

2-15. (canceled)

16. The method according to claim 1, wherein antibody is capable of depleting the population of CD163+ tumor associated macrophages.

17. The method according to claim 1, wherein the method is for enhancing the potency/efficacy of an immune checkpoint inhibitor.

18. The method of claim 1, wherein the cancer is melanoma.

19. The method according to claim 1, wherein the cancer is resistant to immune checkpoint therapy.

20. The method according to claim 1, wherein the antibody binds to the extracellular domain of CD163.

21. The method according to claim 1, wherein the antibody mediates antibody-dependent cell-mediated cytotoxicity.

22. The method according to claim 1, wherein the antibody is an antibody-drug conjugate.

23. A method of treating a cancer in a subject in need thereof comprising administering to the subject a therapeutically effective combination comprising at least one immune checkpoint inhibitor and an antibody having binding affinity for CD163.

24. The method according to claim 23, wherein the method leads to the depletion of CD163+ TAMs in the subject's tumor.

25. The method of claim 23, wherein the immune checkpoint inhibitor is selected from the group consisting of PD-1 antagonists, PD-L1 antagonists, PD-L2 antagonists, CTLA-4 antagonists, VISTA antagonists, TIM-3 antagonists, LAG-3 antagonists, IDO antagonists, KIR2D antagonists, A2AR antagonists, B7-H3 antagonists, B7-H4 antagonists, and BTLA antagonists.

26. The method of claim 23, wherein the cancer is melanoma.

27. The method according to claim 23, wherein the antibody binds to the extracellular domain of CD163.

28. The method according to claim 23, wherein the antibody mediates antibody-dependent cell-mediated cytotoxicity.

29. The method according to claim 23, wherein the antibody is an antibody-drug conjugate.

30. The method according to claim 23, wherein the method is for enhancing the potency/efficacy of the immune checkpoint inhibitor.

31. The method according to claim 23, wherein the cancer is resistant to immune checkpoint therapy.

32. A method of preventing resistance to an administered immune checkpoint inhibitor in a subject suffering from a cancer comprising administering to the subject a therapeutically effective amount of an antibody having binding affinity for CD163.

33. The method according to claim 32, wherein the antibody having binding affinity for CD163 is capable of depleting the population of CD163+ tumor associated macrophages.


SEQUENCE LISTING (1 sequence)

SEQ ID NO: 1 - protein, 1156 aa, Homo sapiens
MSKLRMVLLEDSGSADFRRHFVNLSPFTITVVLLLSACFVTSSLGGTDKELRLVDGENKCSGRVEVKVQEEWGTVCNNGWSMEAVSVICNQLGCPTAIKAPGWANSSAGSGRIWMDHVSCRGNESALWDCKHDGWGKHSNCTHQQDAGVTCSDGSNLEMRLTRGGNMCSGRIEIKFQGRWGTVCDDNFNIDHASVICRQLECGSAVSFSGSSNFGEGSGPIWFDDLICNGNESALWNCKHQGWGKHNCDHAEDAGVICSKGADLSLRLVDGVTECSGRLEVRFQGEWGTICDDGWDSYDAAVACKQLGCPTAVTAIGRVNASKGFGHIWLDSVSCQGHEPAIWQCKHHEWGKHYCNHNEDAGVTCSDGSDLELRLRGGGSRCAGTVEVEIQRLLGKVCDRGWGLKEADVVCRQLGCGSALKTSYQVYSKIQATNTWLFLSSCNGNETSLWDCKNWQWGGLTCDHYEEAKITCSAHREPRLVGGDIPCSGRVEVKHGDTWGSICDSDFSLEAASVLCRELQCGTVVSILGGAHFGEGNGQIWAEEFQCEGHESHLSLCPVAPRPEGTCSHSRDVGVVCSRYTEIRLVNGKTPCEGRVELKTLGAWGSLCNSHWDIEDAHVLCQQLKCGVALSTPGGARFGKGNGQIWRHMFHCTGTEQHMGDCPVTALGASLCPSEQVASVICSGNQSQTLSSCNSSSLGPTRPTIPEESAVACIESGQLRLVNGGGRCAGRVEIYHEGSWGTICDDSWDLSDAHVVCRQLGCGEAINATGSAHFGEGTGPIWLDEMKCNGKESRIWQCHSHGWGQQNCRHKEDAGVICSEFMSLRLTSEASREACAGRLEVFYNGAWGTVGKSSMSETTVGVVCRQLGCADKGKINPASLDKAMSIPMWVDNVQCPKGPDTLWQCPSSPWEKRLASPSEETWITCDNKIRLQEGPTSCSGRVEIWHGGSWGTVCDDSWDLDDAQVVCQQLGCGPALKAFKEAEFGQGTGPIWLNEVKCKGNESSLWDCPARRWGHSECGHKEDAAVNCTDISVQKTPQKATTGRSSRQSSFIAVGILGVVLLAIFVALFFLTKKRRQRQRLAVSSRGENLVHQIQYREMNSCLNADDLDLMNSSENSHESADFSAAELISVSKFLPISGMEKEAILSHTEKENGNL